(19) United States
(12) Patent Application Publication
(10) Pub. No.: US 2003/0194721 A1
(43) Pub. Date: Oct. 16, 2003

(54) Genes expressed in treated foam cells

(75) Inventors: Thomas Mikita, San Francisco, CA (US); Dov Shiffman, Palo Alto, CA (US); J. Gordon Porter, Newark, CA (US); Matthew R. Kaser, Castro Valley, CA (US)

Correspondence Address: INCYTE GENOMICS, INC., 3160 PORTER DRIVE, PALO ALTO, CA, 94304, US

(73) Assignee: Incyte Genomics, Inc., Palo Alto, CA (US)

(21) Appl. No.: 10/247,671

(22) Filed: Sep. 18, 2002

Related U.S. Application Data
(60) Provisional application No. 60/323,784, filed on Sep. 19, 2001.

(51) Int. Cl. C12Q 1/68; G01N 33/53; C07H 21/04; C12P 21/02; C12N 9/00; C12N 5/06

(52) U.S. Cl. 435/6; 435/7.1; 435/69.1; 435/70.21; 435/183; 435/320.1; 435/325; 536/23.2

(57) ABSTRACT

The invention relates to isolated polynucleotides, purified polypeptides, and compositions comprising pluralities of polynucleotides that are differentially expressed when foam cells are treated with oxidized low-density lipoprotein and LPS as associated with atherosclerosis. The invention also presents the use of the polynucleotides as elements on a substrate and provides methods for using the polynucleotides and polypeptides.

This application claims benefit of provisional application Serial No. 60/323,784, filed Sep. 19, 2001.

### FIELD OF THE INVENTION

The present invention relates to a combination of polynucleotides which may be used to detect the differential expression of genes in human foam cells treated with oxidized low-density lipoprotein and/or lipopolysaccharide (LPS). In particular, the combination may be used in the diagnosis of conditions, diseases, and disorders associated with cardiovascular disease.

### BACKGROUND OF THE INVENTION

Atherosclerosis and the associated coronary artery disease and cerebral stroke represent the most common cause of death in industrialized nations. Although certain key risk factors have been identified, a full molecular characterization that elucidates the causes and provides care for these associated diseases has not been achieved. Molecular characterization of growth and regression of atherosclerotic vascular lesions requires identification of the genes that contribute to formation of the lesion including growth, stability, dissolution, rupture and, most lethally, induction of occlusive vessel thrombus.

An early step in the development of atherosclerosis is formation of the “fatty streak”. Lipoproteins, such as the cholesterol-rich low-density lipoprotein (LDL), accumulate in the extracellular space of the vascular intima, and undergo modification. Oxidation of LDL occurs most avidly in the sub-endothelial space where circulating antioxidant defenses are less effective. The degree of LDL oxidation affects its interaction with target cells. “Minimally oxidized” LDL (MM-LDL) is able to bind to LDL receptor but not to the oxidized LDL (Ox-LDL) or “scavenger” receptors that have been identified, including scavenger receptor types A and B, CD36, CD68/macrosialin, and LOX-1 (Navab et al. (1994) Arterioscler Thromb Vasc Biol 16:831-842; Kodama et al. (1990) Nature 343:531-535; Acton et al. (1994) J Biol Chem 269:21003-21009; Endemann et al. (1993) J Biol Chem 268:11811-11816; Ramprasad et al. (1996) Proc Natl Acad Sci 92:14833-14838; and Kataoka et al. (1999) Circulation 99:3110-3117). MM-LDL can increase the adherence and penetration of monocytes, stimulate the release of monocyte chemotactic protein 1 (MCP-1) by endothelial cells, and induce scavenger receptor A (SRA) and CD36 expression in macrophages (Cushing et al. (1990) Proc Natl Acad Sci 87:5134-5138; Yoshida et al. (1998) Arterioscler Thromb Vasc Biol 18:794-802; and Steinberg (1997) J Biol Chem 272:20963-20966). SRA and the other scavenger receptors can bind Ox-LDL and enhance uptake of lipoprotein particles.

Mononuclear phagocytes enter the intima, differentiate into macrophages, and ingest modified lipids including Ox-LDL. In most cell types, cholesterol content is tightly controlled by feedback regulation of LDL receptors and biosynthetic enzymes (Brown and Goldstein (1986) Science 232:34-47). In macrophages, however, the additional scavenger receptors lead to unregulated uptake of cholesterol (Brown and Goldstein (1983) Annu Rev Biochem 52:223-261) and accumulation of multiple intracellular lipid droplets producing a “foam cell” phenotype. Cholesterol-engorged and dead macrophages contribute most of the mass of early “fatty streak” plaques and typical “advanced” lesions of diseased arteries. Numerous studies have described a variety of foam cell responses that contribute to growth and rupture of atherosclerotic vessel wall plaques. These responses include production of multiple growth factors and cytokines, which promote proliferation and adherence of neighboring cells; chemokines, which further attract circulating monocytes into the growing plaque; proteins, which remodel the extracellular matrix; and tissue factor, which can trigger thrombosis (Ross (1993) Nature 362:801-809; Quin et al. (1987) Proc Natl Acad Sci 84:2995-2998). Thus, cholesterol-loaded macrophages which occur in abundance in most stages of the atherosclerotic plaque formation contribute to inception of the atherosclerotic process and to eventual plaque rupture and occlusive thrombus.

During Ox-LDL uptake, macrophages produce cytokines and growth factors that elicit further cellular events that modulate atherogenesis such as smooth muscle cell proliferation and production of extracellular matrix. Additionally, these macrophages may activate genes involved in inflammation including inducible nitric oxide synthase.

In vivo, infectious agents like cytomegalovirus and Chlamydia pneumoniae have been linked to atherosclerosis (Boer et al. (2000) J Pathol 190:237-243; Temesgen (2001) Expert Opin Pharmacother 2:765-772). For example, proinflammatory stimuli such as LPS or tumor necrosis factor-alpha (TNF-α) activate endothelial cells (EC) and inhibit EC apoptosis through induction of nuclear factor kappa B (NF-κB)-dependent genes, such as antiapoptotic gene A20, whereas OxLDL suppresses such induction (Heermeier et al. (2001) J Am Soc Nephrol 12:456-463). These and other studies implicate a role for activated macrophages and other inflammatory cells in the development and progression of atherosclerosis. Thus, genes differentially expressed during foam cell stimulation may reasonably be expected to be markers of the atherosclerotic process.

The invention provides for a combination comprising a plurality of polynucleotides for use in detecting changes in expression of genes encoding proteins that are associated with cardiovascular disorders. The invention satisfies a need in the art by providing a combination of polynucleotides that represent differentially expressed genes which may be used entirely or in part to diagnose, to stage, to treat, or to monitor the progression or treatment of a subject with a cardiovascular vascular disorder.

### SUMMARY OF THE INVENTION

The invention provides a combination comprising a plurality of polynucleotides wherein the polynucleotides have the nucleic acid sequences of SEQ ID NOs:1-127, and the complements of SEQ ID NOs:1-127, that are differentially expressed following treatment of the foam cells with LPS. The invention also presents a combination comprising a plurality of polynucleotides wherein the polynucleotides are SEQ ID NOs:16-105 and 108-127 that are early markers of foam cell formation and the complements of SEQ ID NOs:16-105 and 108-127. The invention further presents a combination comprising a plurality of polynucleotides wherein the polynucleotides are SEQ ID NOs:16-105 and 108-127 that are upregulated following treatment with LPS and the complements of SEQ ID NOs:16-105 and 108-127. The invention still further presents a combination comprising a plurality of polynucleotides wherein the polynucleotides are SEQ ID NOs:1-15 and 106-107 that are downregulated following treatment with LPS and the complements of SEQ ID NOs:1-15 and 106-107. In one aspect, a combination is immobilized on a substrate. In another aspect, a combination is useful to diagnose a cardiovascular disorder.

The invention provides a high throughput method to detect differential expression of one or more of the polynucleotides of the combination. The method comprises hybridizing a substrate containing the combination with the nucleic acids of a sample, thereby forming one or more hybridization complexes, detecting the hybridization complexes so formed, and comparing the hybridization complexes with those of a standard, wherein differences in the size and signal intensity of each hybridization complex indicate differential expression of nucleic acids in the sample. In one aspect, the nucleic acids of the sample are amplified before hybridization. In another aspect, the sample is from a subject with a cardiovascular disorder and differential expression determines the stage of the disorder.

The invention provides a high throughput method for using a combination comprising a plurality of polynucleotides to screen a library or a plurality of molecules or compounds to identify a molecule or compound which specifically binds each polynucleotide of the combination, the method comprising contacting a substrate comprising the combination with a library or a plurality of molecules or compounds under conditions to allow specific binding and detecting specific binding between a molecule or compound and each polynucleotide, thereby identifying a molecule or compound that specifically binds each polynucleotide. In one aspect, the library or plurality of molecules or compounds is selected from DNA molecules, peptides, proteins, and RNA molecules. The invention also provides a method for using a combination comprising a plurality of polynucleotides to identify and purify a ligand, the method comprising contacting a substrate containing the combination with a sample under conditions which allow specific binding between a ligand and each polynucleotide and separating each polynucleotide from its ligand, thereby identifying and obtaining purified ligand.

The invention provides an isolated polynucleotide encoding a protein having an amino acid sequence selected from SEQ ID NOs:154 and 155. The invention also provides an isolated polynucleotide selected from SEQ ID NOs:51, 52, 54, 79, 85, 102, 106, and 119 and the complements of SEQ ID NOs:51, 52, 54, 79, 85, 102, 106, and 119. The invention further provides an expression vector containing the polynucleotide, a host cell containing the expression vector, and a method for producing a protein comprising culturing the host cell under conditions for the expression of protein and recovering the protein from the host cell culture.

The invention provides a purified protein comprising an amino acid sequence selected from SEQ ID NOs:154 or 155, a biologically active fragment of SEQ ID NOs:154 or 155, and an antigenic epitope selected from SEQ ID NOs:154 or 155. In one alternative, the invention provides an isolated protein comprising the amino acid sequence of SEQ ID NO:154. In another alternative, the invention provides an isolated protein comprising the amino acid sequence of SEQ ID NO:155. The invention also provides a high-throughput method for screening a library or plurality of molecules or compounds to identify at least one ligand which specifically binds a protein, the method comprising contacting the protein with the molecules or compounds under conditions to allow specific binding and detecting specific binding, thereby identifying a ligand which specifically binds the protein. The library or plurality of molecules or compounds are selected from agonists, antagonists, antibodies, DNA molecules, small molecule drugs, immunoglobulins, inhibitors, mimetics, peptide nucleic acids, peptides, pharmaceutical agents, proteins, RNA molecules, and ribozymes. The invention further provides a method for using a protein to purify a ligand, the method comprising combining the protein or a portion thereof with a sample under conditions to allow specific binding, recovering the bound protein, and separating the protein from the ligand, thereby obtaining purified ligand. The invention still further provides a method for using the protein to produce an antibody, the method comprising immunizing an animal with the protein or an antigenic determinant thereof under conditions to elicit an antibody response, isolating animal antibodies, and screening the isolated antibodies with the protein to identify an antibody which specifically binds the protein. The invention yet still further provides a method for using the protein to purify antibodies which specifically bind to the protein.

The invention provides a purified antibody. The invention also provides a method of using an antibody to detect the expression of a protein in a sample, the method comprising contacting the antibody with a sample under conditions for the formation of an antibody:protein complex and detecting complex formation wherein the formation of the complex indicates the expression of the protein in the sample. In one aspect, complex formation is compared to standards and is diagnostic of a cardiovascular disorder. The invention further provides using an antibody to immunopurify a protein comprising combining the antibody with a sample under conditions to allow formation of an antibody:protein complex, and separating the antibody from the protein, thereby obtaining purified protein. The invention provides a composition comprising a polynucleotide, a protein, an antibody, or a ligand which has agonistic or antagonistic activity.

### DESCRIPTION OF THE FIGURES AND TABLES

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

The Sequence Listing is a compilation of polynucleotides and their encoded polypeptides obtained by sequencing clone inserts (isolates) of different cDNAs and identified by hybrid complex formation using the cDNAs as probes on a microarray. Each sequence is identified by a sequence identification number (SEQ ID NO) and by an Incyte ID number. The Incyte ID number represents the gene sequence that contains the clone insert.

FIGS. 1A, 1B, and 1C show the protein (SEQ ID NO:154) encoded by the polynucleotide having the nucleic acid sequence of SEQ ID NO:51 as produced using MACDNASIS PRO software (Hitachi Software Engineering, South San Francisco Calif.).

FIGS. 2A, 2B, 2C, and 2D show the protein (SEQ ID NO:155) encoded by the polynucleotide having the nucleic acid sequence of SEQ ID NO:54 as produced using MACDNASIS PRO software (Hitachi Software Engineering).

Table 1 shows the differentially expressed genes associated with foam cells treated with LPS identified by cluster analysis.

Table 2 shows a identification (ID) map for each polynucleotide and its encoded polypeptide.

### DETAILED DESCRIPTION OF THE INVENTION

Definitions

“Antibody” refers to intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment; and an antibody-peptide fusion protein.

“Antigenic determinant” refers to an antigenic or immunogenic epitope, structural feature, or region of an oligopeptide, peptide, or protein which is capable of inducing formation of an antibody which specifically binds the protein. Biological activity is not a prerequisite for immunogenicity.

“Array” refers to an ordered arrangement of at least two polynucleotides, proteins, or antibodies on a substrate. At least one of the polynucleotides, proteins, or antibodies represents a control or standard, and the other polynucleotide, protein, or antibody of diagnostic or therapeutic interest. The arrangement of at least two and up to about 40,000 polynucleotides, proteins, or antibodies on the substrate assures that the size and signal intensity of each labeled complex, formed between each polynucleotide and at least one nucleic acid, each protein and at least one ligand or antibody, or each antibody and at least one protein to which the antibody specifically binds, is individually distinguishable.

“Cancer” includes an adenocarcinoma, leukemia, lymphoma, melanoma, myeloma, sarcoma, teratocarcinoma, and cancers of the adrenal gland, bladder, bone, bone marrow, brain, breast, cervix, colon, esophagus, gall bladder, ganglia, heart, kidney, liver, lung, muscle, ovary, pancreas, parathyroid, penis, pituitary gland, prostate, salivary glands, skin, small intestine, spleen, stomach, testis, thymus, thyroid, and uterus

A “combination” comprises at least two sequences selected from SEQ ID NOs:1-127 and their complements as presented in the Sequence Listing.

“Polynucleotide” refers to an isolated polynucleotide, cDNA, or nucleic acid sequence that may have originated recombinantly or synthetically, be double-stranded or single-stranded, represents coding and noncoding 3′ or 5′ sequence, generally lacks introns and may be purified or combined with carbohydrate, lipids, protein or inorganic elements or substances as a useful composition.

The phrase “polynucleotide encoding a protein” refers to a nucleic acid whose sequence closely aligns with sequences that encode conserved regions, motifs or domains identified by employing analyses well known in the art. These analyses include BLAST (Basic Local Alignment Search Tool; Altschul (1993) J Mol Evol 36:290-300; Altschul et al. (1990) J Mol Biol 215:403-410) and BLAST2 (Altschul et al. (1997) Nucleic Acids Res 25:3389-3402) which provide identity within the conserved region. Brenner et al. (1998; Proc Natl Acad Sci 95:6073-6078) who analyzed BLAST for its ability to identify structural homologs by sequence identity found 30% identity is a reliable threshold for sequence alignments of at least 150 residues and 40% is a reasonable threshold for alignments of at least 70 residues (Brenner, page 6076, column 2).

A “composition” refers to the polynucleotide and a labeling moiety; a purified protein and a pharmaceutical carrier or a heterologous, labeling or purification moiety; an antibody and a labeling moiety or pharmaceutical agent; and the like.

“Derivative” refers to a polynucleotide or a protein that has been subjected to a chemical modification. Derivatization of a polynucleotide can involve substitution of a nontraditional base such as queosine or of an analog such as hypoxanthine. These substitutions are well known in the art. Derivatization of a protein involves the replacement of a hydrogen by an acetyl, acyl, alkyl, amino, formyl, or morpholino group. Derivative molecules retain the biological activities of the naturally occurring molecules but may confer longer lifespan or enhanced activity.

“Differential expression” refers to an increased or upregulated or a decreased or downregulated expression as detected by absence, presence, or at least two-fold change in the amount of transcribed messenger RNA or translated protein in a sample.

“Disorder” refers to cardiovascular conditions, diseases or syndromes including disorders of increased vascularization such as cancer including hemangioma and hemangioendothelioma, diabetic retinopathy, follicular cysts, Kaposi's sarcoma, endometriosis, obesity and peritoneal sclerosis; disorders of insufficient vascularization such as atherosclerosis and ulcers; and disorders of abnormal remodeling such as psoriasis.

An “expression profile” is a representation of gene expression in a sample. A nucleic acid expression profile is produced using sequencing, hybridization, or amplification (quantitative PCR) technologies and mRNAs or cDNAs from a sample. A protein expression profile, although time delayed, mirrors the nucleic acid expression profile and may use antibody or protein arrays, enzyme-linked immunosorbent assays, fluorescence-activated cell sorting, spatial immobilization such as 2D-PAGE in conjunction with a scintillation counter, mass spectrophotometry, or western analysis or affinity chromatography, to detect protein expression in a sample. The nucleic acids, proteins, or antibodies may be used in solution or attached to a substrate, and their detection is based on methods and labeling moieties well known in the art. Expression profiles may also be evaluated by methods such as electronic northern analysis, guilt-by-association, and transcript imaging. Expression profiles produced using any of the above methods may be contrasted with expression profiles produced using normal or diseased tissues. Of note is the correspondence between mRNA and protein expression has been discussed by Zweiger (2001, Transducing the Genome. McGraw-Hill, San Francisco, Calif.) and Glavas et al. (2001; T cell activation upregulates cyclic nucleotide phosphodiesterases 8A1 and 7A3, Proc Natl Acad Sci 98:6319-6342) among others.

“Fragment” refers to a chain of consecutive nucleotides from about 60 to about 5000 base pairs in length. Fragments may be used in PCR, hybridization or array technologies to identify related nucleic acids and in binding assays to screen for a ligand. Such ligands are useful as therapeutics to regulate replication, transcription or translation.

A “hybridization complex” is formed between a polynucleotide and a nucleic acid of a sample when the purines of one molecule hydrogen bond with the pyrimidines of the complementary molecule, e.g., 5′-A-G-T-C-3′ base pairs with 3′-T-C-A-G-5′. The degree of complementarity and the use of nucleotide analogs affect the efficiency and stringency of hybridization reactions.

“Identity” as applied to sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul (1997) supra). BLAST2 may be used in a standardized and reproducible way to insert gaps in one of the sequences in order to optimize alignment and to achieve a more meaningful comparison between them. “Similarity” as applied to proteins uses the same algorithms but takes into account conservative substitutions of nucleotides or residues.

“Isolated” or “purified” refers to any molecule or compound that is separated from its natural environment and is from about 60% free to about 90% free from other components with which it is naturally associated.

“Labeling moiety” refers to any reporter molecule whether a visible or radioactive label, stain or dye that can be attached to or incorporated into a polynucleotide or protein. Visible labels and dyes include but are not limited to anthocyanins, β glucuronidase, BIODIPY, Coomassie blue, Cy3 and Cy5, digoxigenin, FITC, green fluorescent protein, luciferase, spyro red, silver, and the like. Radioactive markers include radioactive forms of hydrogen, iodine, phosphorous, sulfur, and the like.

“Ligand” refers to any agent, molecule, or compound which will bind specifically to a complementary site on a cDNA molecule or polynucleotide, or to an epitope or a protein. Such ligands stabilize or modulate the activity of polynucleotides or proteins and may be composed of inorganic or organic substances including nucleic acids, proteins, carbohydrates, fats, and lipids.

“Oligonucleotide” refers a single stranded molecule from about 18 to about 60 nucleotides in length which may be used in hybridization or amplification technologies or in regulation of replication, transcription or translation. Equivalent terms include amplimer, primer, and oligomer.

“Portion” refers to any part of a protein used for any purpose which retains at least one biological or antigenic characteristic of a native protein, but especially, to an epitope for the screening of ligands or for production of antibodies.

“Post-translational modification” of a protein can involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like.

“Probe” refers to a polynucleotide that hybridizes to at least one nucleic acid in a sample. Where targets are single stranded, probes are complementary single strands. Probes can be labeled for use in hybridization reactions including Southern, northern, in situ, dot blot, array, and like technologies or in screening assays.

“Protein” refers to a polypeptide or any portion thereof. An “oligopeptide” is an amino acid sequence from about five residues to about 15 residues that is used as part of a fusion protein to produce an antibody.

“Sample” is used in its broadest sense as containing nucleic acids, proteins, antibodies, and the like. A sample may comprise a bodily fluid; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue or tissue biopsy; a tissue print; buccal cells, skin, a hair or its follicle; and the like.

“Specific binding” refers to a special and precise interaction between two molecules which is dependent upon their structure, particularly their molecular side groups. For example, the intercalation of a regulatory protein into the major groove of a DNA molecule, the hydrogen bonding along the backbone between two single stranded nucleic acids, or the binding between an epitope of a protein and an agonist, antagonist, or antibody.

“Substrate” refers to any rigid or semi-rigid support to which polynucleotides or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” (TI) is a profile of gene transcription activity in a particular tissue at a particular time. TI provides assessment of the relative abundance of expressed polynucleotides in the cDNA libraries of an EST database as described in U.S. Pat. No. 5,840,484, incorporated herein by reference.

“Variant” refers to molecules that are recognized variations of a polynucleotide or a protein encoded by the polynucleotide. Splice variants may be determined by BLAST score, wherein the score is at least 100, and most preferably at least 400. Allelic variants have a high percent identity to the polynucleotides and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid.

The Invention

The invention provides a combination comprising a plurality of polynucleotides, wherein each polynucleotide is differentially expressed in foam cells treated with LPS. The plurality of polynucleotides comprise the nucleic acid sequences of SEQ ID NOs:1-127, as presented in the Sequence Listing, or the complements of SEQ ID NOs:1-127. Additionally, the invention provides a subset of polynucleotides whose expression is upregulated, SEQ ID NOs:16-105 and 108-127 or downregulated, SEQ ID NOs:1-15 and 106-107, when foam cells are treated with oxidized low-density lipoprotein and LPS. The nucleotide sequences (SEQ ID NOs:1-127) were used to identify open reading frames within the sequences in the LIFESEQ databases (Incyte Genomics).

The invention also provides novel polypeptides comprising the amino acid sequences of SEQ ID NOs:154 and 155 encoded by polynucleotides having the nucleic acid sequences of SEQ ID NOs:51 and 54, respectively. The nucleic acid and amino acid sequences were queried against databases such as the LIFESEQ (Incyte), GenBank, and SwissProt databases using BLAST. Motifs, HMM algorithms, and alignments with BLOCKS, PRINTS, PROFILESCAN, Prosite, and PFAM databases were used to perform functional analyses; the antigenic index (Jameson-Wolf analysis) was determined using LASERGENE software (DNASTAR).

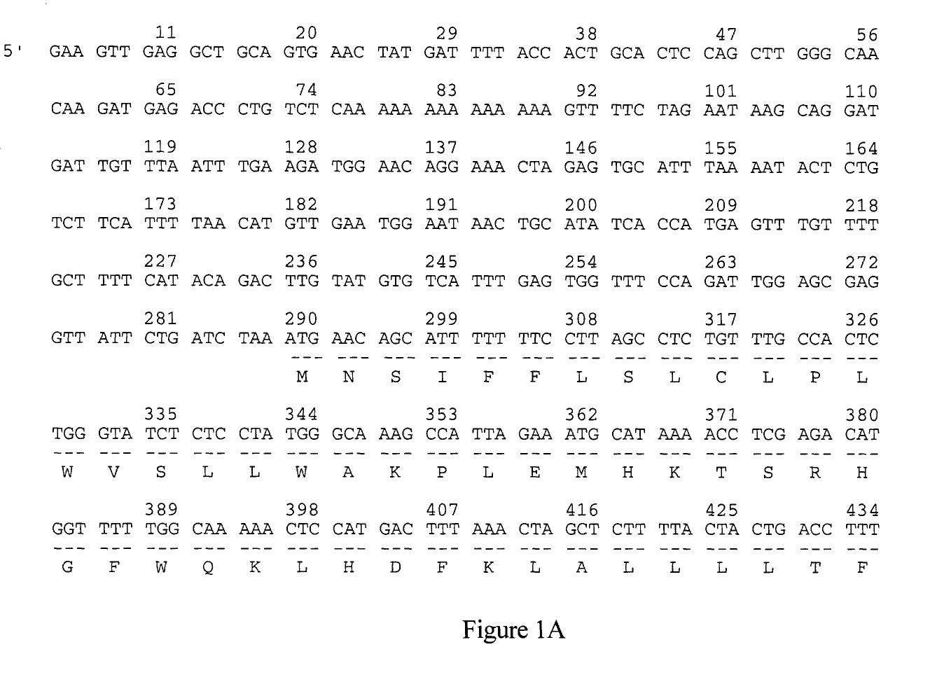

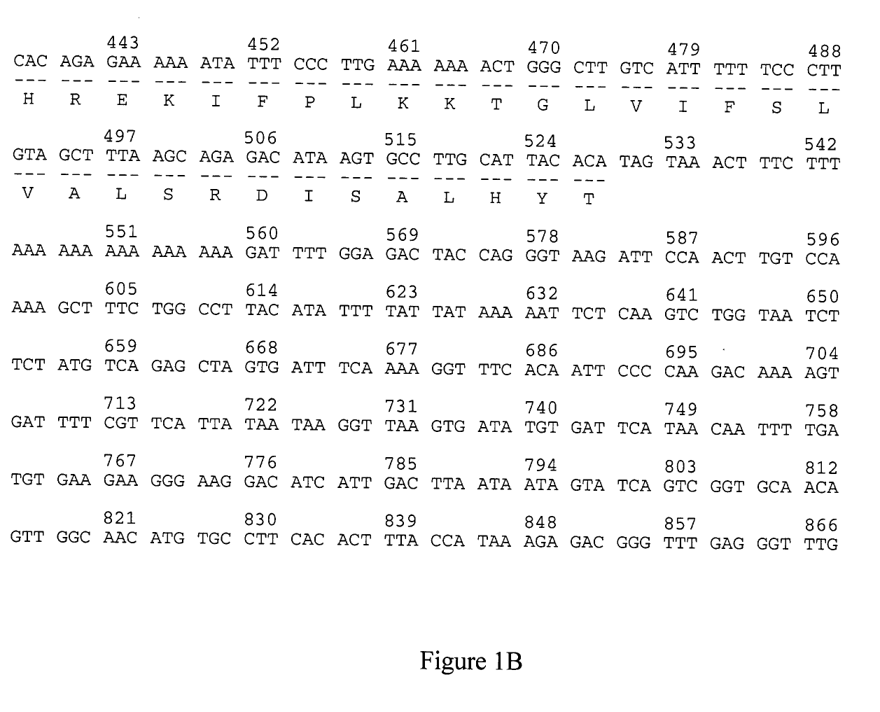

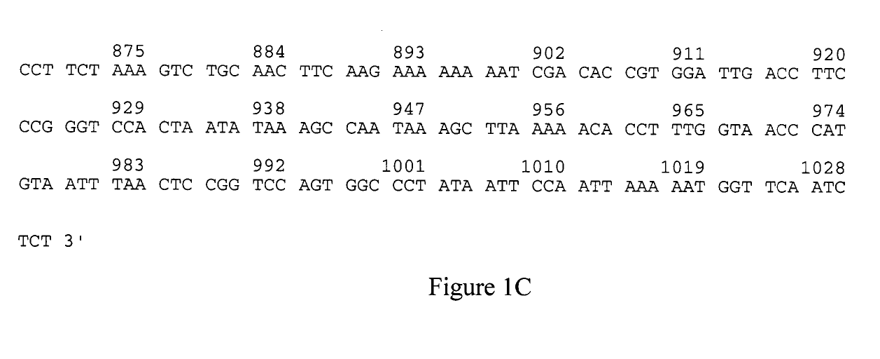

Translation of SEQ ID NO:51 using MACDNASIS PRO software (Hitachi Software Engineering) and default parameters is shown in FIGS. 1A-1C for the protein having the amino acid sequence of SEQ ID NO:154. Functional analysis identified a GATA-type zinc finger domain from residues C10 through T48, Ets-domain signature and profile from residues A20 through L67, fork head domain signature and profile from residues M1 through A76, MADS-box domain signature and profile from residues A20 through H78, (PROFILESCAN); an Ets-domain from residues H30 through G61, a Sigma-54 transcription factor family signature from residues K26 through 154, an zinc finger C2H2 type signature from residues C9 through H38 (BLIMPS); and a predicted transmembrane domain from residues S3 through H31 (TMAP). The oligopeptide portion of SEQ ID NO:154 from residues L23 through H31 is useful as an antigenic fragment.

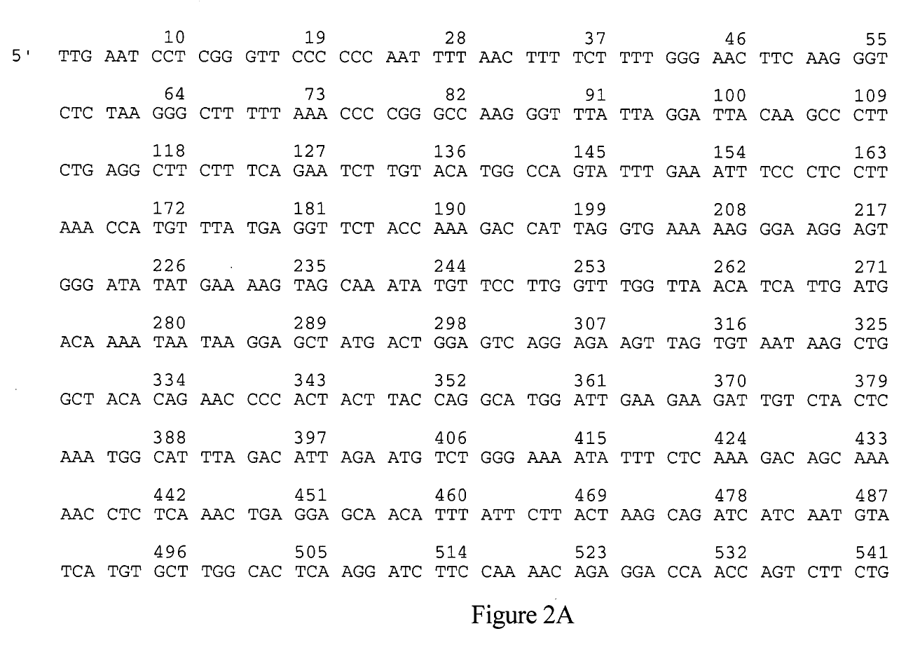

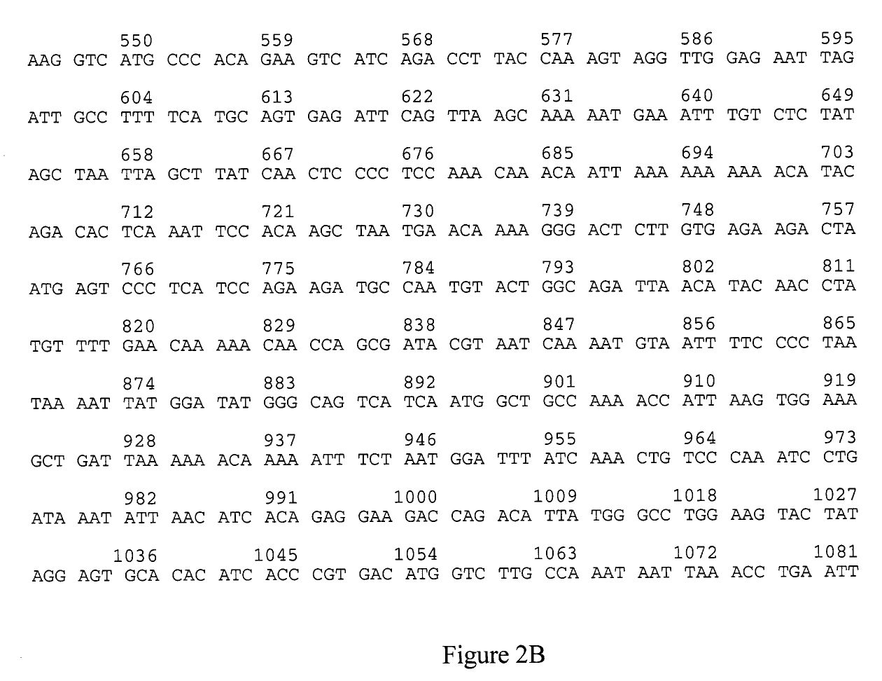

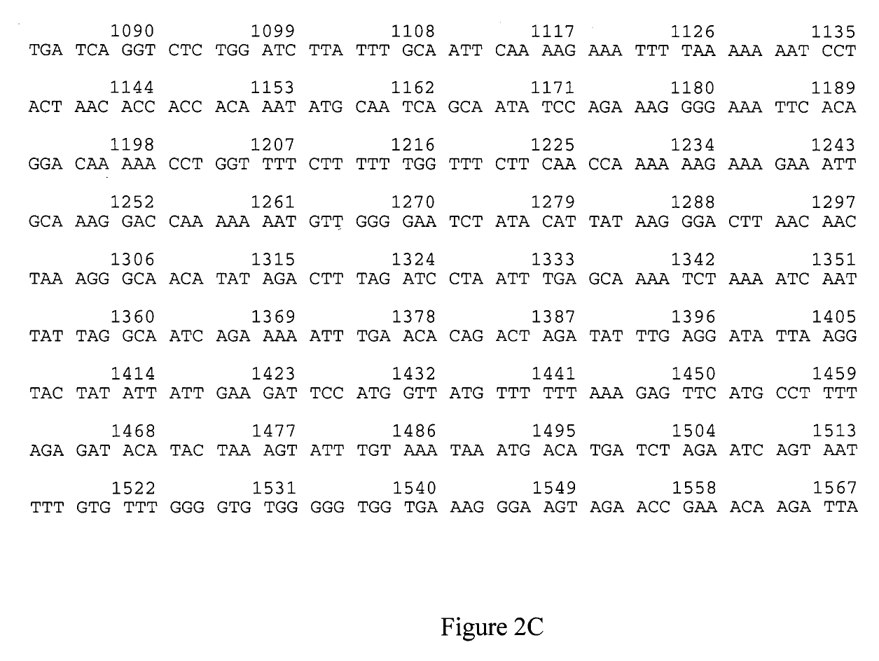

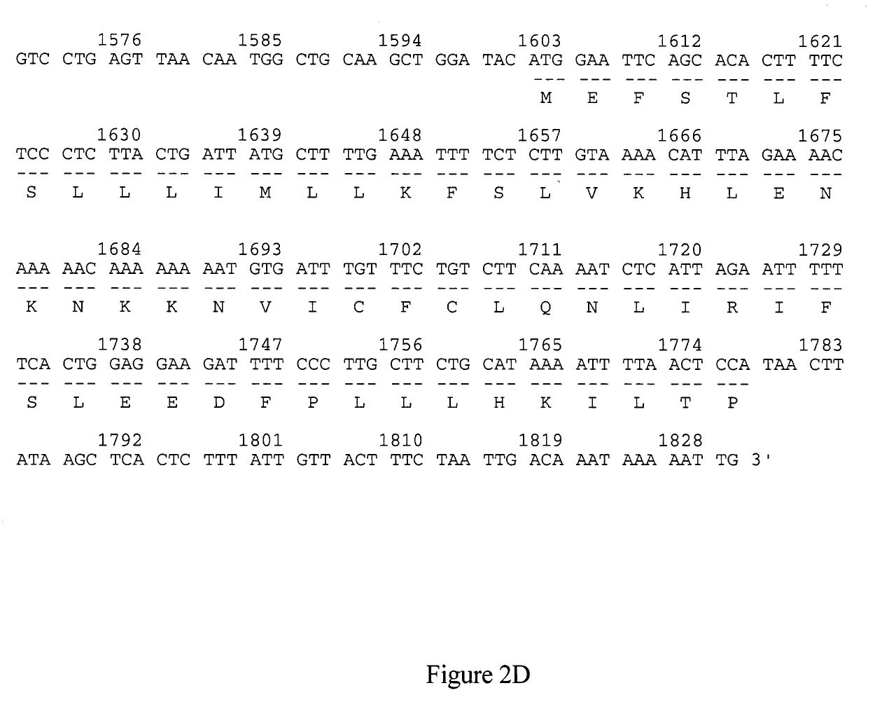

Translation of SEQ ID NO:54 using MACDNASIS PRO software (Hitachi Software Engineering) and default parameters is shown in FIGS. 2A-2D for the protein having the amino acid sequence of SEQ ID NO:155. Functional analysis of SEQ ID NO:155 using BLOCKS, PRINTS, PROFILESCAN, Prosite, PFAM, Motifs, and HMM algorithms identified an Ets-domain signature and profile from M1 through L51, a MADS-box domain signature and profile from residues E2 through E47 (PROFILESCAN); and a predicted transmembrane domain from residues 4S through V20 (TMAP). The oligopeptide portion of SEQ ID NO:155 from residues K21 through V31 is useful as an antigenic fragment.

Method for Selecting Polynucleotides

Human THP-1 cells (American Type Culture Collection (ATCC), Manassas Va.) were grown in serum-containing medium and differentiated with 12-0-tetradecanoyl-phorbol-13-acetate (Research Biochemical International (RBI), Natick Mass.) for 24 hours. Cells were then cultured either in the presence or absence (control) of Ox-LDL for 3 days. The Ox-LDL pretreated and control cells were then activated by treatment with LPS. Poly(A) RNA from cultured cells was prepared for expression profiling after 0, 1, and 6 hours exposure to LPS. Poly(A) RNAs from experimental and control cells were labeled with Cy3 and Cy5 fluorescent dyes and hybridized in time-matched pairs on UNIGEM V 2.0 and HumanGenome GEMI arrays (Incyte Genomics, Palo Alto Calif.).

Agglomerative cluster analysis was used to identify response patterns and to establish relationships between different gene expression profiles. Each measurement was normalized by dividing the expression ratios by the maximum value for each time series. The clustering process defined a hierarchical tree with the number of branches intersecting at each branch level of the tree equal to the number of clusters at that level. Division of the tree at branch level 5 divides the 127 differentially expressed polynucleotides into 14 clusters.

Table 1 shows the differentially expressed gene transcripts and splice variants associated with foam cell development identified by cluster analysis. Column 1 shows the SEQ ID NO, column 2 shows the Incyte ID number, and column 3 shows the sequence annotation. Columns 4 through 10 show normalized differential expression based on the data converted from fold increase or decrease to log₂ such that an 2-fold increase reads “1.0” or a 2-fold decrease reads “−1.0”. LPS effects on transcription were determined in the control cells by comparing 1 hr and 6 hr RNA samples to time 0 levels (data designated: 1 h−/0− and 6 h−/0−; Table 1, columns 5 and 6, respectively). LPS effects on transcription in the OxLDL pre-treated cells were similarly determined (data designated: 1 hr+/0+ and 6 hr+/0+; Table 1, columns 7 and 8, respectively). OxLDL effects on LPS responsive genes were determined through matched time point comparisons of RNA from control and OxLDL pre-treated cells after LPS addition (data designated: 1 h+/1 h− and 6 h+/6 h−; Table 1, columns 9 and 10, respectively).

Table 2 shows a ID map for each polynucleotide and its encoded polypeptide. Column 1 shows the polynucleotide SEQ ID NO and column 2 shows the SEQ ID NO of the encoded polypeptide.

In particular, SEQ ID NOs:1-15 organize into co-expressing clusters with one another as shown by the shading patterns in Table 1. Similarly, SEQ ID NOs:16-17; SEQ ID NOs:18-25; SEQ ID NOs:26-38; SEQ ID NOs:39-63; SEQ ID NO:64; SEQ ID NOs:65-70; SEQ ID NOs:71-99; SEQ ID NOs:100-105; SEQ ID NOs:106; SEQ ID NOs:107-108; SEQ ID NOs:109-111; SEQ ID NOs:112-126; and SEQ ID NO:127 organize into thirteen co-expressing clusters as shown by the shading patterns in Table 1.

In particular, SEQ ID NO:51 (2303994CB1) co-expresses with sequences encoding chemokine receptors, cytokine receptors, and cytokine-inducible proteins. Therefore SEQ ID NO:51 is predicted to encode chemokine- or cytokine-inducible protein or an inflammation-related second messenger pathway protein. In particular, the polypeptide sequence of SEQ ID NO:154 is encoded by SEQ ID NO:51.

In particular, SEQ ID NO:52 (376673.3) co-expresses with sequences encoding chemokine receptors, cytokine receptors, and cytokine-inducible proteins. Therefore SEQ ID NO:52 is predicted to encode chemokine- or cytokine-inducible protein or an inflammation-related second messenger pathway protein.

In particular, SEQ ID NO:54 (1554082CB1) co-expresses with sequences encoding chemokine receptors, cytokine receptors, and cytokine-inducible proteins. Therefore SEQ ID NO:54 is predicted to encode chemokine- or cytokine-inducible protein or an inflammation-related second messenger pathway protein. In particular, the polypeptide sequence of SEQ ID NO:155 is encoded by SEQ ID NO:54.

In particular, SEQ ID NO:79 (234681.21) co-expresses with sequences encoding cytokine-inducible proteins, cytokine receptors, and cytokine-inducible transcription factors. Therefore SEQ ID NO:79 is predicted to encode cytokine-inducible protein or an inflammation-related second messenger pathway protein.

In particular, SEQ ID NO:85 (898547.1) co-expresses with sequences encoding cytokine-inducible proteins, cytokine receptors, and cytokine-inducible transcription factors. Therefore SEQ ID NO:85 is predicted to encode cytokine-inducible protein or an inflammation-related second messenger pathway protein.

In particular, SEQ ID NO:102 (1447015.4) co-expresses with sequences encoding transcription factors, nuclear receptors, and methyl-CpG binding proteins. Therefore SEQ ID NO:102 is predicted to encode a DNA-binding protein.

In particular, SEQ ID NO:119 (128475.1) co-expresses with sequences encoding the human tumor necrosis factor a inducible protein A20, transcription factors, cytokines, and protein tyrosine phosphatase proteins. Therefore SEQ ID NO:119 is predicted to encode cytokine protein or an inflammation-related second messenger pathway protein.

In particular, expression of SEQ ID NO:106 (277161.34) was downregulated at both 1 hour and 6 hours following LPS-treatment in both control and Ox-LDL-loaded cells. Therefore SEQ ID NO:106 is useful as a control nucleotide sequence in an analysis of foam cell inflammatory response, a specific utility.

In particular, SEQ ID NOs:4, 5, 6, 7, 8, 9, 12, 15, 17, 19, 21, 22, 23, 24, 25, 26, 27, 28, 30, 32, 33, 34, 35, 42, 45, 47, 51, 54, 55, 58, 61, 62, 74, 75, 76, 81, 83, 87, 88, 91, 92, 93, 94, 95, 96, 97, 100, 101, 107, 108, 111, 112, 114, 116, 120, 121, 123, 124, and 125 have open reading frames encoding polypeptide sequences. As shown in Table 2, the polynucleotides of SEQ ID NOs:4, 5, 6, 7, 8, 9, 12, 15, 17, 19, 21, 22, 23, 24, 25, 26, 27, 28, 30, 32, 33, 34, 35, 42, 45, 47, 51, 54, 55, 58, 61, 62, 74, 75, 76, 81, 83, 87, 88, 91, 92, 93, 94, 95, 96, 97, 100, 101, 107, 108, 111, 112, 114, 116, 120, 121, 123, 124, and 125, encode the polypeptides of SEQ ID NOs:128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, and 186, respectively.

The polynucleotides of the invention can be genomic DNA, cDNA, mRNA, or any RNA-like or DNA-like material such as peptide nucleic acids (PNA), branched DNAs and the like. Polynucleotide probes can be sense or antisense strand. Where targets are double stranded, probes may be either sense or antisense strands. Where targets are single stranded, probes are complementary single strands. In one embodiment, polynucleotides are cDNAs. In another embodiment, polynucleotides are plasmids. In the case of plasmids, the sequence of interest is the cDNA insert.

Polynucleotides can be prepared by a variety of synthetic or enzymatic methods well known in the art. Polynucleotides can be synthesized, in whole or in part, using chemical methods well known in the art (Caruthers et al. (1980) Nucleic Acids Symp Ser (7)215-233). Alternatively, polynucleotides can be produced enzymatically or recombinantly, by in vitro or in vivo transcription.

Nucleotide analogs can be incorporated into polynucleotide probes by methods well known in the art. The only requirement is that the incorporated nucleotide analogs of the probe must base pair with target nucleotides. For example, certain guanine nucleotides can be substituted with hypoxanthine which base pairs with cytosine residues. However, these base pairs are less stable than those between guanine and cytosine. Alternatively, adenine nucleotides can be substituted with 2,6-diaminopurine which can form stronger base pairs with thymidine than those between adenine and thymidine. Additionally, polynucleotides can include nucleotides that have been derivatized chemically or enzymatically. Typical chemical modifications include derivatization with acyl, alkyl, aryl or amino groups.

Polynucleotides can be synthesized on a substrate. Synthesis on the surface of a substrate may be accomplished using a chemical coupling procedure and a piezoelectric printing apparatus as described by Baldeschweiler et al. (PCT publication WO95/251116). Alternatively, the polynucleotides can be synthesized on a substrate surface using a self-addressable electronic device that controls when reagents are added as described by Heller et al. (U.S. Pat. No. 5,605,662; incorporated herein by reference).

The polynucleotide or the cDNA can be immobilized on a substrate by covalent means such as by chemical bonding procedures or UV irradiation. In one such method, a cDNA is bound to a glass surface which has been modified to contain epoxide or aldehyde groups. In another case, a probe is placed on a polylysine coated surface and then UV cross-linked as described by Shalon et al. (WO95/35505). In yet another method, a DNA is actively transported from a solution to a given position on a substrate by electrical means (Heller, supra). Alternatively, polynucleotides, clones, plasmids or cells can be arranged on a membrane or filter. In the latter case, cells are lysed, proteins and cellular components degraded, and the DNA is coupled to the membrane or filter by UV cross-linking.

Furthermore, polynucleotides do not have to be directly bound to the substrate, but rather can be bound to the substrate through a linker group. The linker groups are typically about 6 to 50 atoms long to provide exposure of the attached probe. Preferred linker groups include ethylene glycol oligomers, diamines, diacids and the like. Reactive groups on the substrate surface react with a terminal group of the linker to bind the linker to the substrate. The other terminus of the linker is then bound to the polynucleotide.

Polynucleotides can be attached to a substrate by sequentially dispensing reagents for probe synthesis on the substrate surface or by dispensing preformed DNA fragments to the substrate surface. Typical dispensers include a micropipette delivering solution to the substrate with a robotic system to control the position of the micropipette with respect to the substrate. There can be a multiplicity of dispensers delivering the reagents to their assigned reaction region on the array.

Use of the Polynucleotides

The polynucleotide of the invention may be used for a variety of purposes. For example, the combination of the invention may be used as elements on a microarray. The microarray can be used in high-throughput methods such as for detecting a related polynucleotide in a sample, screening libraries of molecules or compounds to identify a ligand, or diagnosing a particular cardiovascular condition, disease, or disorder and particularly atherosclerosis. Alternatively, a polynucleotide complementary to a given sequence of the sequence listing can be used to inhibit or inactivate a therapeutically relevant gene related to the polynucleotide.

When the combination of the invention is employed as elements on a microarray, the polynucleotide elements are organized in an ordered fashion so that each element is present at a specified location on the substrate. Because the elements are at specified locations on the substrate, the hybridization patterns and intensities, which together create a unique expression profile, can be interpreted in terms of expression levels of particular genes and can be correlated with a particular metabolic process, condition, disorder, disease, stage of disease, or treatment.

Hybridization

The polynucleotides or fragments or complements thereof of the invention may be used in various hybridization technologies. The polynucleotides may be naturally occurring, recombinant, or chemically synthesized; based on genomic or cDNA sequences; and labeled using a variety of reporter molecules by either PCR or enzymatic techniques. Commercial kits are available for labeling and cleanup of such polynucleotides or probes. Radioactive, fluorescent, and chemiluminescent labeling, are well known in the art. Alternatively, a polynucleotide is cloned into a commercially available vector, and probes are produced by transcription. The probe is synthesized and labeled by addition of an appropriate polymerase, such as T7 or SP6 polymerase, and at least one labeled nucleotide.

A probe may be designed or derived from unique regions of the polynucleotide, such as the 3′ untranslated region or from a conserved motif, and used in protocols to identify naturally occurring molecules encoding the same polypeptide, allelic variants, or related molecules. The probe may be DNA or RNA, is usually single stranded and should have at least 50% sequence identity to any of the nucleic acid sequences. The probe may comprise at least 18 contiguous nucleotides of a polynucleotide. Such a probe may be used under hybridization conditions that allow binding only to an identical sequence or under conditions that allow binding to a related sequence with at least one nucleotide substitution or deletion. Related sequences may be discovered using a pool of degenerate probes and appropriate hybridization conditions. Generally, a probe for use in Southern or northern hybridizations may be from about 400 to about 4000 nucleotides long. Such probes may be single-stranded or double-stranded and may have high binding specificity in solution-based or substrate-based hybridizations. A probe may also be an oligonucleotide that is used to detect a polynucleotide of the invention in a sample by PCR.

The stringency of hybridization is determined by G+C content of the probe, salt concentration, and temperature. In particular, stringency is increased by reducing the concentration of salt or raising the hybridization temperature. In solutions used for some membrane-based hybridizations, addition of an organic solvent such as formamide allows the reaction to occur at a lower temperature. Hybridization may be performed with buffers, such as 5×saline sodium citrate (SSC) with 1% sodium dodecyl sulfate (SDS) at 60° C., that permits the formation of a hybridization complex between nucleic acid sequences that contain some mismatches. Subsequent washes are performed with buffers such as 0.2×SSC with 0.1% SDS at either 45° C. (medium stringency) or 650-68° C. (high stringency). At high stringency, hybridization complexes will remain stable only where the polynucleotides are completely complementary. In some membrane-based hybridizations, preferably 35% or most preferably 50%, formamide may be added to the hybridization solution to reduce the temperature at which hybridization is performed. Background signals may be reduced by the use of detergents such as Sarkosyl or TRITON X-100 (Sigma-Aldrich, St. Louis Mo.) and a blocking agent such as denatured salmon sperm DNA. Selection of components and conditions for hybridization are well known to those skilled in the art and are reviewed in Ausubel et al. (1997, Short Protocols in Molecular Biology, John Wiley & Sons, New York N.Y., pp. 6.11-6.19, 14.11-14.36, and A1-43).

Dot-blot, slot-blot, low density and high density arrays are prepared and analyzed using methods known in the art. Probes or array elements from about 18 consecutive nucleotides to about 5000 consecutive nucleotides are contemplated by the invention and used in array technologies. The array may be used to monitor the expression level of large numbers of genes simultaneously and to identify genetic variants, mutations, and SNPs. Such information may be used to determine gene function; to understand the genetic basis of a disorder; to diagnose a disorder; and to develop and monitor the activities of therapeutic agents being used to control or cure a disorder. (See, e.g., U.S. Pat. No. 5,474,796; PCT application WO95/11995; PCT application WO95/35505; U.S. Pat. Nos. 5,605,662; and 5,958,342.)

Screening Assays

A polynucleotide may be used to screen a library or a plurality of molecules or compounds for a ligand with specific binding affinity. The ligands may be DNA molecules, peptides, proteins, and RNA molecules and include such molecules as transcription factors, enhancers, or repressors that regulate the activity, replication, transcription, or translation of the polynucleotide in the biological system. The assay involves combining the polynucleotide or a fragment thereof with the molecules or compounds under conditions that allow specific binding and detecting the bound polynucleotide to identify at least one ligand that specifically binds the polynucleotide.

In one embodiment, the polynucleotide of the invention may be incubated with a library of isolated and purified molecules or compounds and binding activity determined by methods well known in the art, e.g., a gel-retardation assay (U.S. Pat. No. 6,010,849) or a reticulocyte lysate transcriptional assay. In another embodiment, the polynucleotide may be incubated with nuclear extracts from biopsied and/or cultured cells and tissues. Specific binding between the polynucleotide and a molecule or compound in the nuclear extract is initially determined by gel shift assay and may be later confirmed by raising antibodies against that molecule or compound. When these antibodies are added into the assay, they cause a supershift in the gel-retardation assay.

In another embodiment, the polynucleotide may be used to purify a molecule or compound using affinity chromatography methods well known in the art. In one embodiment, the polynucleotide is chemically reacted with cyanogen bromide groups on a polymeric resin or gel. Then a sample is passed over and reacts with or binds to the polynucleotide. The molecule or compound which is bound to the polynucleotide may be released from the polynucleotide by increasing the salt concentration of the flow-through medium and collected.

Purification of Ligand

The polynucleotide or a fragment thereof may be used to purify a ligand from a sample. A method for using a mammalian polynucleotide or a fragment thereof to purify a ligand would involve combining the polynucleotide or a fragment thereof with a sample under conditions to allow specific binding, recovering the bound polynucleotide, and using an appropriate agent to separate the polynucleotide from the purified ligand.

Protein Production and Uses

The polynucleotides of this application or their full length cDNAs may be used to produce purified polypeptides using recombinant DNA technologies described herein and taught in Ausubel (supra; pp. 16.1-16.62). One of the advantages of producing polypeptides by these procedures is the ability to obtain highly-enriched sources of the polypeptides thereby simplifying purification procedures. The invention also encompasses amino acid substitutions, deletions or insertions made on the basis of similarity in polarity, charge, solubility, hydrophobicity, hydrophilicity, and/or the amphipathic nature of the residues involved. Such substitutions may be conservative in nature when the substituted residue has structural or chemical properties similar to the original residue (e.g., replacement of leucine with isoleucine or valine) or they may be nonconservative when the replacement residue is radically different (e.g., a glycine replaced by a tryptophan). Computer programs included in LASERGENE software (DNASTAR, Madison Wis.), MACVECTOR software (Genetics Computer Group, Madison Wis.) and RasMol software (University of Massachusetts, Amherst Mass.) may be used to help determine which and how many amino acid residues in a particular portion of the polypeptide may be substituted, inserted, or deleted without abolishing biological or immunological activity.

Expression of Encoded Proteins

Expression of a particular polynucleotide may be accomplished by cloning the polynucleotide into an appropriate vector and transforming this vector into an appropriate host cell. The cloning vector used for the construction of the human libraries may also be used for expression. Such vectors usually contain a promoter and a polylinker useful for cloning, priming, and transcription. An exemplary vector may also contain the promoter for β-galactosidase, an amino-terminal methionine and the subsequent seven amino acid residues of β-galactosidase. The vector may be transformed into an appropriate host strain of E. coli. Induction of the isolated bacterial strain with isopropylthiogalactoside (IPTG) using standard methods will produce a fusion protein that contains an N terminal methionine, the first seven residues of β-galactosidase, about 15 residues of linker, and the polypeptide encoded by the polynucleotide.

The polynucleotide may be shuttled into other vectors known to be useful for expression of protein in specific hosts. Oligonucleotides containing cloning sites and fragments of DNA sufficient to hybridize to stretches at both ends of the polynucleotide may be chemically synthesized by standard methods. These primers may then be used to amplify the desired fragments by PCR. The fragments may be digested with appropriate restriction enzymes under standard conditions and isolated using gel electrophoresis. Alternatively, similar fragments are produced by digestion of the polynucleotide with appropriate restriction enzymes and filled in with chemically synthesized oligonucleotides. Fragments of the coding sequence from more than one gene may be ligated together and expressed.

Signal sequences that dictate secretion of soluble proteins are particularly desirable as component parts of a recombinant sequence. For example, a chimeric protein may be expressed that includes one or more additional purification-facilitating domains. Such domains include, but are not limited to, metal-chelating domains that allow purification on immobilized metals, protein A domains that allow purification on immobilized immunoglobulin, and the domain utilized in the FLAGS extension/affinity purification system (Immunex, Seattle Wash.). The inclusion of a cleavable-linker sequence such as ENTEROKINASEMAX (Invitrogen, San Diego Calif.) between the polypeptide and the purification domain may also be used to recover the polypeptide.

Suitable expression hosts may include, but are not limited to, mammalian cells such as Chinese Hamster Ovary (CHO) and human 293 cells, insect cells such as Sf9 cells, yeast cells such as Saccharomyces cerevisiae, and bacteria such as E. coli. For each of these cell systems, a useful expression vector may also include an origin of replication and one or two selectable markers to allow selection in bacteria as well as in a transfected eukaryotic host. Vectors for use in eukaryotic expression hosts may require the addition of 3′ poly(A) tail if the polynucleotide lacks poly(A).

Additionally, the vector may contain promoters or enhancers that increase gene expression. Most promoters are host specific, and they include MMTV, SV40 or metallothionein promoters for CHO cells; trp, lac, tac or T7 promoters for bacterial hosts; or alpha factor, alcohol oxidase or PGH promoters for yeast. Adenoviral vectors with enhancers such as the rous sarcoma virus (RSV) enhancer or retroviral vectors with promoters such as the long terminal repeat (LTR) promoter may be used to drive protein expression in mammalian cell lines. Once homogeneous cultures of recombinant cells are obtained, large quantities of a secreted soluble polypeptide may be recovered from the conditioned medium and analyzed using chromatographic methods well known in the art. An alternative method for the production of large amounts of secreted protein involves the transformation of mammalian embryos and the recovery of the recombinant protein from milk produced by transgenic cows, goats, sheep, and the like.

In addition to recombinant production, polypeptides or portions thereof may be produced using solid-phase techniques (Stewart et al. (1969) Solid-Phase Peptide Synthesis, W H Freeman, San Francisco Calif.; Merrifield (1963) J Am Chem Soc 5:2149-2154), manually, or using machines such as the 431A Peptide synthesizer (Amersham Biosciences (APB), Piscataway N.J.). Polypeptides produced by any of the above methods may be used as pharmaceutical compositions to treat disorders associated with underexpression.

Screening Assays

A protein or a portion thereof encoded by the polynucleotide may be used to screen libraries or a plurality of molecules or compounds for a ligand with specific binding affinity or to purify a molecule or compound from a sample. The polypeptide or portion thereof employed in such screening may be free in solution, affixed to an abiotic or biotic substrate, or located intracellularly. For example, viable or fixed prokaryotic host cells that are stably transformed with recombinant nucleic acids that have expressed and positioned a polypeptide on their cell surface can be used in screening assays. The cells are screened against libraries or a plurality of ligands and the specificity of binding or formation of complexes between the expressed polypeptide and the ligand may be measured. The ligands may be DNA, RNA, or PNA molecules, agonists, antagonists, antibodies, immunoglobulins, inhibitors, peptides, pharmaceutical agents, proteins, drugs, or any other test molecule or compound that specifically binds the polypeptide. An exemplary assay involves combining the mammalian polypeptide or a portion thereof with the molecules or compounds under conditions that allow specific binding and detecting the bound polypeptide to identify at least one ligand that specifically binds the polypeptide.

This invention also contemplates the use of competitive drug screening assays in which neutralizing antibodies capable of binding the polypeptide specifically compete with a test compound capable of binding to the polypeptide or oligopeptide or fragment thereof. One method for high throughput screening using very small assay volumes and very small amounts of test compound is described in U.S. Pat. No. 5,876,946. Molecules or compounds identified by screening may be used in a mammalian model system to evaluate their toxicity, diagnostic, or therapeutic potential.

Purification of a Ligand

The polypeptide or a portion thereof may be used to purify a ligand from a sample. A method for using a mammalian polypeptide or a portion thereof to purify a ligand would involve combining the polypeptide or a portion thereof with a sample under conditions to allow specific binding, recovering the bound polypeptide, and using an appropriate chaotropic agent to separate the polypeptide from the purified ligand.

Production of Antibodies

A polypeptide encoded by a polynucleotide of the invention may be used to produce specific antibodies. Antibodies may be produced using an oligopeptide or a portion of the polypeptide with inherent immunological activity. Methods for producing antibodies include: 1) injecting an animal (usually goats, rabbits, or mice) with the polypeptide, or a portion or an oligopeptide thereof, to induce an immune response; 2) engineering hybridomas to produce monoclonal antibodies; 3) inducing in vivo production in the lymphocyte population; or 4) screening libraries of recombinant immunoglobulins. Recombinant immunoglobulins may be produced as taught in U.S. Pat. No. 4,816,567.

Antibodies produced using the polypeptides of the invention are useful for the diagnosis of prepathologic disorders as well as the diagnosis of chronic or acute diseases characterized by abnormalities in the expression, amount, or distribution of the polypeptide. A variety of protocols for competitive binding or immunoradiometric assays using either polyclonal or monoclonal antibodies specific for polypeptides are well known in the art. Immunoassays typically involve the formation of complexes between a polypeptide and its specific binding molecule or compound and the measurement of complex formation.

Immunoassay procedures may be used to quantify expression of the polypeptide in cell cultures, in subjects with a particular disorder or in model animal systems under various conditions. Increased or decreased production of polypeptides as monitored by immunoassay may contribute to knowledge of the cellular activities associated with developmental pathways, engineered conditions or diseases, or treatment efficacy. The quantity of a given polypeptide in a given tissue may be determined by performing immunoassays on freeze-thawed detergent extracts of biological samples and comparing the slope of the binding curves to binding curves generated by purified polypeptide.

Labeling of Molecules for Assay

A wide variety of reporter molecules and conjugation techniques are known by those skilled in the art and may be used in various polynucleotide, polypeptide or antibody arrays or assays. Synthesis of labeled molecules may be achieved using Promega (Madison Wis.) or APB kits for incorporation of a labeled nucleotide such as ³²P-dCTP, Cy3-dCTP or Cy5-dCTP or amino acid such as ³⁵S-methionine. Polynucleotides, polypeptides, or antibodies may be directly labeled with a reporter molecule by chemical conjugation to amines, thiols and other groups present in the molecules using reagents such as BIODIPY or FITC (Molecular Probes, Eugene Oreg.).

The polypeptides and antibodies may be labeled for purposes of assay by joining them, either covalently or noncovalently, with a reporter molecule that provides for a detectable signal. A wide variety of labels and conjugation techniques are known and have been reported in the scientific and patent literature including, but not limited to U.S. Pat. Nos. 3,817,837; 3,850,752; 3,939,350; 3,996,345; 4,277,437; 4,275,149; and 4,366,241.

Diagnostics

The polynucleotides, or fragments thereof, may be used to detect and quantify differential gene expression; absence, presence, or excess expression of mRNAs; or to monitor mRNA levels during therapeutic intervention. Cardiovascular disorders associated with differential expression particularly include atherosclerosis and associated complications. These polynucleotides can also be utilized as markers of treatment efficacy against the disorders noted above over a period ranging from several days to months. The diagnostic assay may use hybridization or amplification technology to compare gene expression in a biological sample from a patient to standard samples in order to detect differential gene expression. Methods for this comparison are elucidated herein and are well known in the art.

For example, the polynucleotide may be labeled by standard methods and added to a biological sample from a patient under conditions for the formation of hybridization complexes. After an incubation period, the sample is washed and the amount of label (or signal) associated with hybridization complexes, is quantified and compared with a standard value. If the amount of label in the patient sample is significantly altered in comparison to the standard value, then the presence of the disorder is indicated.

In order to provide a basis for the diagnosis of a condition, disease or disorder associated with gene expression, a normal or standard expression profile is established. This may be accomplished by combining a biological sample taken from normal subjects, either animal or human, with a probe under conditions for hybridization or amplification. Standard hybridization may be quantified by comparing the values obtained using normal subjects with values from an experiment in which a known amount of a purified target sequence is used. Standard values obtained in this manner may be compared with values obtained from samples from patients who are symptomatic for a particular condition, disease, or disorder. Deviation from standard values toward those associated with a particular condition is used to diagnose that condition.

Such assays may also be used to evaluate the efficacy of a particular therapeutic treatment regimen in animal studies and in clinical trial or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment.

Gene Expression Profiles

A gene expression profile comprises a plurality of polynucleotides and a plurality of detectable hybridization complexes, wherein each complex is formed by hybridization of one or more probes to one or more complementary sequences in a sample. The combination of the invention is used as elements on a microarray to analyze gene expression profiles. In one embodiment, the microarray is used to monitor the progression of disease. Researchers can assess and catalog the differences in gene expression between healthy and diseased tissues or cells. By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, the microarray is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

In another embodiment, animal models which mimic a human disease can be used to characterize expression profiles associated with a particular condition, disorder or disease or treatment of the condition, disorder or disease. Novel treatment regimens may be tested in these animal models using microarrays to establish and then follow expression profiles over time. In addition, microarrays may be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

Assays Using Antibodies

Antibodies directed against epitopes on a protein encoded by a polynucleotide of the invention may be used in assays to quantify the amount of protein found in a particular human cell. Such assays include methods utilizing the antibody and a label to detect expression level under normal or disease conditions. The antibodies may be used with or without modification, and labeled by joining them, either covalently or noncovalently, with a labeling moiety.

Protocols for detecting and measuring protein expression using either polyclonal or monoclonal antibodies are well known in the art. Examples include ELISA, RIA, and fluorescent activated cell sorting (FACS). Such immunoassays typically involve the formation of complexes between the protein and its specific antibody and the measurement of such complexes. Preferred methods may employ a two-site, monoclonal-based immunoassay utilizing monoclonal antibodies reactive to two non-interfering epitopes or a competitive binding assay. (See, e.g., Coligan et al. (1997) Current Protocols in Immunology, Wiley-Interscience, New York N.Y.; Pound (1998) Immunochemical Protocols, Humana Press, Totowa N.J.).

Therapeutics

The polynucleotides of the invention can be used in gene therapy via delivery to a target tissue, such as mononuclear phagocytes. Expression of the protein encoded by the polynucleotide may correct a disorder associated with reduction or loss of endogenous target protein. Polynucleotides may be delivered to specific cells in vitro; and the transformed cells transferred in vivo to various tissues. Alternatively, polynucleotides may be delivered in vivo. Polynucleotides are delivered to cells or tissues using vectors such as retrovirus, adenovirus, adeno-associated virus, herpes simplex virus, and bacterial plasmids. Non-viral methods of gene delivery include cationic liposomes, polylysine conjugates, artificial viral envelopes, and direct injection of DNA (Anderson (1998) Nature 392:25-30; Dachs et al. (1997) Oncol Res 9:313-325; Chu et al. (1998) J Mol Med 76(3-4):184-192; August et al. (1997) Gene Therapy (Advances in Pharmacology Vol. 40), Academic Press, San Diego Calif.).

In addition, expression of a particular protein can be modulated through the specific binding of an antisense polynucleotide sequence to a nucleic acid sequence which either encodes the protein or directs its expression. The antisense polynucleotide can be DNA, RNA, branched or single-stranded nucleic acids with or with analog modifications. The nucleic acid sequence can be cellular mRNA and/or genomic DNA and binding of the antisense sequence can affect transcription and/or translation, respectively. Antisense sequences can be delivered intracellularly using viral vectors or non-viral vectors as described above or by Weiss et al. (1999; Cell Mol Life Sci 55(3):334-358) or Agrawal (1996; Antisense Therapeutics, Humana Press, Totowa N.J.).

Both polynucleotides and antisense sequences can be produced ex vivo by using any nucleic acid synthesizers or other automated systems known in the art. Polynucleotides and antisense sequences can also be produced biologically by transforming an appropriate host cell with an expression vector containing the sequence of interest.

Molecules which modulate the expression of a polynucleotide of the invention or activity of the encoded protein are useful as therapeutics for conditions and disorders associated with an immune response. Such molecules include agonists which increase the expression or activity of the polynucleotide or encoded protein, respectively; or antagonists which decrease expression or activity of the polynucleotide or encoded protein, respectively. In one aspect, an antibody which specifically binds the protein may be used directly as an antagonist or indirectly as a targeting or delivery mechanism for bringing a pharmaceutical agent to cells or tissues which express the protein.

Additionally, any of the proteins or their ligands, or complementary nucleic acid sequences may be administered in combination with other appropriate therapeutic agents. Selection of the appropriate agents for use in combination therapy may be made by one of ordinary skill in the art, according to conventional pharmaceutical principles. The combination of therapeutic agents may act synergistically to affect the treatment or prevention of the conditions and disorders associated with an immune response. Using this approach, one may be able to achieve therapeutic efficacy with lower dosages of each agent, thus reducing the potential for adverse side effects. Further, the therapeutic agents may be combined with pharmaceutically-acceptable carriers including excipients and auxiliaries which facilitate processing of the active compounds into preparations which can be used pharmaceutically. Further details on techniques for formulation and administration may be found in the latest edition of Remington's Pharmaceutical Sciences (Mack Publishing, Easton Pa.).

Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of underexpression or overexpression of genes of interest and for the development of methods for diagnosis and treatment of diseases. A mammal inbred to overexpress a particular gene (for example, secreted in milk) may also serve as a convenient source of the protein expressed by that gene.

Transgenic Animal Models

Transgenic rodents that overexpress or underexpress a gene of interest may be inbred and used to model human diseases or to test therapeutic or toxic agents. (See, e.g., U.S. Pat. Nos. 5,175,383 and 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Embryonic Stem Cells

Embryonic (ES) stem cells isolated from rodent embryos retain the potential to form embryonic tissues. When ES cells are placed inside a carrier embryo, they resume normal development and contribute to tissues of the live-born animal. ES cells are the preferred cells used in the creation of experimental knockout and knockin rodent strains. Mouse ES cells, such as the mouse 129/SvJ cell line, are derived from the early mouse embryo and are grown under culture conditions well known in the art. Vectors used to produce a transgenic strain contain a disease gene candidate and a marker gene, the latter serves to identify the presence of the introduced disease gene. The vector is transformed into ES cells by methods well known in the art, and transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains.

ES cells derived from human blastocysts may be manipulated in vitro to differentiate into at least eight separate cell lineages. These lineages are used to study the differentiation of various cell types and tissues in vitro, and they include endoderm, mesoderm, and ectodermal cell types that differentiate into, for example, neural cells, hematopoietic lineages, and cardiomyocytes.

Knockout Analysis

In gene knockout analysis, a region of a gene is enzymatically modified to include a non-natural intervening sequence such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene. Transformed cells are injected into rodent blastulae, and the blastulae are implanted into pseudopregnant dams. Transgenic progeny are crossbred to obtain homozygous inbred lines that lack a functional copy of the mammalian gene.

Knockin Analysis

ES cells can be used to create knockin humanized animals (pigs) or transgenic animal models (mice or rats) of human diseases. With knockin technology, a region of a human gene is injected into animal ES cells, and the human sequence integrates into the animal cell genome. Transformed cells are injected into blastulae and the blastulae are implanted as described above. Transgenic progeny or inbred lines are studied and treated with potential pharmaceutical agents to obtain information on treatment of the analogous human condition. These methods have been used to model several human diseases.

As described herein, the uses of the polynucleotides, provided in the Sequence Listing of this application, and their encoded polypeptides are exemplary of known techniques and are not intended to reflect any limitation on their use in any technique that would be known to the person of average skill in the art. Furthermore, the polynucleotides provided in this application may be used in molecular biology techniques that have not yet been developed, provided the new techniques rely on properties of nucleotide sequences that are currently known to the person of ordinary skill in the art, e.g., the triplet genetic code, specific base pair interactions, and the like. Likewise, reference to a method may include combining more than one method for obtaining or assembling full length polynucleotides that will be known to those skilled in the art.

It is to be understood that the invention is not limited to the particular methodology, protocols, and reagents described, as these may vary. It is also understood that the terminology used herein is for the purpose of describing particular embodiments only and is not intended to limit the scope of the present invention which will be limited only by the appended claims. The examples below are provided to illustrate the subject invention and are not included for the purpose of limiting the invention.

### EXAMPLES

I. Construction of cDNA Libraries

RNA was purchased from Clontech Laboratories (Palo Alto Calif.) or isolated from various tissues. Some tissues were homogenized and lysed in guanidinium isothiocyanate, while others were homogenized and lysed in phenol or in a suitable mixture of denaturants, such as TRIZOL reagent (Invitrogen). The resulting lysates were centrifuged over CsCl cushions or extracted with chloroform. RNA was precipitated with either isopropanol or ethanol and sodium acetate, or by other routine methods.

Phenol extraction and precipitation of RNA were repeated as necessary to increase RNA purity. In most cases, RNA was treated with DNAse. For most libraries, poly(A) RNA was isolated using oligo d(T)-coupled paramagnetic particles (Promega), OLIGOTEX latex particles (Qiagen, Valencia Calif.), or an OLIGOTEX mRNA purification kit (Qiagen). Alternatively, poly(A) RNA was isolated directly from tissue lysates using other kits, including the POLY(A)PURE mRNA purification kit (Ambion, Austin Tex.).

In some cases, Stratagene (La Jolla, Calif.) was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen) using the recommended procedures or similar methods known in the art. (See Ausubel, supra, Units 5.1-6.6.) Reverse transcription was initiated using oligo d(T) or random primers. Synthetic oligonucleotide adapters were ligated to double stranded cDNA, and the cDNA was digested with the appropriate-restriction enzyme or enzymes. For most libraries, the cDNA was size-selected (300-1000 bp) using SEPHACRYL S1000, SEPHAROSE CL2B, or SEPHAROSE CL4B column chromatography (APB) or preparative agarose gel electrophoresis. cDNAs were ligated into compatible restriction enzyme sites of the polylinker of the pBLUESCRIPT plasmid (Stratagene), pSPORT1 plasmid (Invitrogen), or pINCY plasmid (Incyte Genomics). Recombinant plasmids were transformed into XL1-Blue, XL1-BlueMRF, or SOLR competent E. coli cells (Stratagene) or DH5α, DH10B, or ELECTROMAX DH10B competent E. coli cells (Invitrogen).

In some cases, libraries were superinfected with a 5×excess of the helper phage, M13K07, according to the method of Vieira et al. (1987, Methods Enzymol 153:3-11) and normalized or subtracted using a methodology adapted from Soares (1994, Proc Natl Acad Sci 91:9228-9232), Swaroop et al. (1991, Nucl Acids Res 19:1954), and Bonaldo et al. (1996, Genome Res 6:791-806). The modified Soares normalization procedure was utilized to reduce the repetitive cloning of highly expressed high abundance cDNAs while maintaining the overall sequence complexity of the library. Modification included significantly longer hybridization times which allowed for increased gene discovery rates by biasing the normalized libraries toward those infrequently expressed low-abundance cDNAs which are poorly represented in a standard transcript image (Soares, supra).

II. Isolation and Sequencing of cDNA Clones

Plasmids were recovered from host cells by in vivo excision using the UNIZAP vector system (Stratagene) or by cell lysis. Plasmids were purified using one of the following: the Magic or WIZARD Minipreps DNA purification system (Promega); the AGTC Miniprep purification kit (Edge BioSystems, Gaithersburg Md.); the QIAWELL 8, QIAWELL 8 Plus, or QIAWELL 8 Ultra plasmid purification systems, or the REAL PREP 96 plasmid purification kit (Qiagen). Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao (1994) Anal Biochem 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes) and a FLUOROSKAN II fluorescence scanner (Labsystems Oy, Helsinki, Finland).

cDNA sequencing reactions were processed using standard methods or high-throughput instrumentation such as the CATALYST 800 thermal cycler (Applied Biosystems (ABI), Foster City Calif.) or the DNA ENGINE thermal cycler (MJ Research, Watertown Mass.) in conjunction with the HYDRA microdispenser (Robbins Scientific, Sunnyvale Calif.) or the MICROLAB 2200 system (Hamilton, Reno Nev.). cDNA sequencing reactions were prepared using reagents provided by APB or supplied in sequencing kits such as the PRISM BIGDYE cycle sequencing kit (ABI). Electrophoretic separation of cDNA sequencing reactions and detection of labeled polynucleotides were carried out using the MEGABACE 1000 DNA sequencing system (APB) or the PRISM 373 or 377 DNA sequencing systems (ABI) in conjunction with standard protocols, base calling software; and sequence analysis systems known in the art. Reading frames within the cDNA sequences were identified using standard methods (reviewed in Ausubel, supra, Unit 7.7).

III. Extension of cDNA Sequences

Nucleic acid sequences were extended using Incyte cDNA clones and oligonucleotide primers. One primer was synthesized to initiate 5′ extension of the known fragment, and the other, to initiate 3′ extension of the known fragment. The initial primers were designed using OLIGO software (Molecular Insights, Cascade, Colo.), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed. Preferred libraries are ones that have been size-selected to include larger cDNAs. Also, random primed libraries are preferred because they will contain more sequences with the 5′ and upstream regions of genes. A randomly primed library is particularly useful if an oligo d(T) library does not yield a full-length cDNA.

High fidelity amplification was obtained by PCR using methods well known in the art. PCR was performed in 96-well plates using the DNA ENGINE thermal cycler (MJ Research). The reaction mix contained DNA template, 200 mmol of each primer, reaction buffer containing Mg²⁺, (NH₄)₂SO₄, and β-mercaptoethanol, Taq DNA polymerase (APB), ELONGASE enzyme (Invitrogen), and Pfu DNA polymerase (Stratagene), with the following parameters for primer pair PCI A and PCI B (Incyte Genomics): Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ (Stratagene) were as follows: Step 1: 94° C., 3 min; Step 2: 94° C., 15 sec; Step 3: 57° C., 1 min;

Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN reagent (0.25% reagent in 1×TE, v/v; Molecular Probes) and 0.5 μl of undiluted PCR product into each well of an opaque fluorimeter plate (Corning Life Sciences, Acton Mass.) and allowing the DNA to bind to the reagent. The plate was scanned in a Fluoroskan II (Labsystems Oy) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended nucleic acids were desalted and concentrated, transferred to 384-well plates, digested with CviJI cholera virus endonuclease (Molecular Biology Research, Madison Wis.), and sonicated or sheared prior to religation into pUC18 vector (APB). For shotgun sequencing, the digested nucleic acids were separated on low concentration (0.6 to 0.8%) agarose gels, fragments were excised, and agar digested with AGARACE enzyme (Promega). Extended clones were religated using T4 DNA ligase (New England Biolabs, Beverly Mass.) into pUC18 vector (APB), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2×carbenicillin liquid media.

The cells were lysed, and DNA was amplified by PCR using Taq DNA polymerase (APB) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified using PICOGREEN reagent (Molecular Probes) as described above. Samples with low DNA recoveries were reamplified using the same conditions described above. Samples were diluted with 20% dimethylsulfoxide (DMSO; 1:2, v/v), and sequenced using DYENAMIC energy transfer sequencing primers and the DYENAMIC DIRECT cycle sequencing kit (APB) or the PRISM BIGDYE terminator cycle sequencing kit (ABI).

IV. Assembly and Analysis of Sequences

The nucleic acid sequences of the polynucleotides presented in the Sequence Listing were prepared by automated methods and may contain occasional sequencing errors and unidentified nucleotides, designated with an N, that reflect state-of-the-art technology at the time the cDNA was sequenced. Vector, linker, and polyA sequences were masked using algorithms and programs based on BLAST, dynamic programming, and dinucleotide nearest neighbor analysis. Ns and SNPs can be verified either by resequencing the cDNA or using algorithms to compare multiple sequences that overlap the area in which the Ns or SNP occur. Both of these techniques are well known to and used by those skilled in the art. The sequences may be analyzed using a variety of algorithms described in Ausubel (1997, unit 7.7) and in Meyers (1995; Molecular Biology and Biotechnology, Wiley VCH, New York N.Y., pp. 856-853).

Component nucleotide sequences from chromatograms were subjected to PHRED analysis (Phil's Revised Editing Program; Phil Green, University of Washington, Seattle Wash.) and assigned a quality score. The sequences having at least a required quality score were subject to various pre-processing algorithms to eliminate low quality 3′ ends, vector and linker sequences, polyA tails, Alu repeats, mitochondrial and ribosomal sequences, bacterial contamination sequences, and sequences smaller than 50 base pairs. Sequences were screened using the BLOCK 2 program (Incyte Genomics), a motif analysis program based on sequence information contained in the SWISS-PROT and PROSITE databases (Bairoch et al. (1997) Nucleic Acids Res 25:217-221; Attwood et al. (1997) J Chem Inf Comput Sci. 37:417-424).

Processed sequences were subjected to assembly procedures in which the sequences were assigned to bins, one sequence per bin. Sequences in each bin were assembled to produce consensus sequences, templates. Subsequent new sequences were added to existing bins using the BLAST (Altschul, 1993, supra; Altschul, 1990, supra; Karlin et al. (1988) Proc Natl Acad Sci 85:841-845), BLASTn (v.1.4, WashU), and CROSSMATCH software (Green, supra). Candidate pairs were identified as all BLAST hits having a quality score greater than or equal to 150. Alignments of at least 82% local identity were accepted into the bin. The component sequences from each bin were assembled using PHRAP (Phil's Revised Alignment Program; Green, supra). Bins with several overlapping component sequences were assembled using DEEP PHRAP (Green, supra).

Bins were compared against each other, and those having local similarity of at least 82% were combined and reassembled. Reassembled bins having templates of insufficient overlap (less than 95% local identity) were re-split. Assembled templates were also subjected to analysis by STITCHER/EXON MAPPER algorithms which analyzed the probabilities of the presence of splice variants, alternatively spliced exons, splice junctions, differential expression of alternative spliced genes across tissue types, disease states, and the like. These resulting bins were subjected to several rounds of the above assembly procedures to generate the template sequences found in the LIFESEQ GOLD database (Incyte Genomics).

The assembled templates were annotated using the following procedure. Template sequences were analyzed using BLASTn (v2.0, NCBI) versus GBpri (GenBank version 109). “Hits” were defined as an exact match having from 95% local identity over 200 base pairs through 100% local identity over 100 base pairs, or a homolog match having an E-value of 1×10⁻⁸. The hits were subjected to frameshift FASTx versus GENPEPT (GenBank version 109). In this analysis, a homolog match was defined as having an E-value of 1×10⁻⁸. The assembly method used above was described in “Database and System for Storing, Comparing and Displaying Related Biomolecular Sequence Information”, U.S. Ser. No. 09/276,534, filed Mar. 25, 1999, incorporated by reference herein, and the LIFESEQ GOLD user manual (Incyte Genomics).

Following assembly, template sequences were subjected to motif, BLAST, Hidden Markov Model (HMM; Pearson and Lipman (1988) Proc Natl Acad Sci 85:2444-2448; Smith and Waterman (1981) J Mol Biol 147:195-197), and functional analyses, and categorized in protein hierarchies using methods described in “Database System Employing Protein Function Hierarchies for Viewing Biomolecular Sequence Data”, U.S. Ser. No. 08/812,290, filed Mar. 6, 1997; “Relational Database for Storing Biomolecule Information”, U.S. Ser. No. 08/947,845, filed Oct. 9, 1997; “Project-Based Full-Length Biomolecular Sequence Database” U.S. Pat. No. 5,953,727; and “Relational Database and System for Storing Information Relating to Biomolecular Sequences”, U.S. Ser. No. 09/034,807, filed Mar. 4, 1998, all of which are incorporated by reference herein. Template sequences may be further queried against public databases such as the GenBank rodent, mammalian, vertebrate, eukaryote, prokaryote, and human EST databases.

V. Preparation of Microarrays

The polynucleotides present on the human UNIGEM V 2.0 and the HumanGenome GEM-I arrays (Incyte Genomics) represent template sequences derived from the LIFESEQ GOLD assembled human sequence database (Incyte Genomics) based on a non-redundant set of gene-oriented clusters derived from IMAGE (integrated molecular analysis of genomes and their expression) cDNA library clones and derived ESTs in the gbEST database (National Center for Biotechnology Information, National Library of Medicine, Bethesda, Md.). A single clone representing each particular template was used on the microarray. Polynucleotides were amplified from bacterial cells using primers complementary to vector sequences flanking the cDNA insert. Thirty cycles of PCR increased the initial quantity of polynucleotide from 1-2 ng to a final quantity greater than 5 μg. Amplified polynucleotides were then purified using SEPHACRYL-400 columns (APB).

Purified polynucleotides were immobilized on polymer-coated glass slides. Glass microscope slides (Corning Life Sciences) were cleaned by ultrasound in 0.1% SDS and acetone, with extensive distilled water washes between and after treatments. Glass slides were etched in 4% hydrofluoric acid (VWR Scientific Products, West Chester Pa.), washed extensively in distilled water, and coated with 0.05% aminopropyl silane (Sigma-Aldrich, St. Louis Mo.) in 95% ethanol. Coated slides were cured in a 110° C. oven. polynucleotides were applied to the coated glass substrate using a procedure described in U.S. Pat. No. 5,807,522, incorporated herein by reference. One microliter of the polynucleotide at an average concentration of 100 ng/ul was loaded into the open capillary printing element by a high-speed robotic apparatus which then deposited about 5 nl of polynucleotide per slide.

Microarrays were UV-crosslinked using a STRATALINKER UV-crosslinker (Stratagene), and then washed at room temperature once in 0.2% SDS and three times in distilled water. Non-specific binding sites were blocked by incubation of microarrays in 0.2% casein in phosphate buffered saline (Tropix, Bedford Mass.) for 30 minutes at 60° C. followed by washes in 0.2% SDS and distilled water as before.

VI. Preparation of Target Polynucleotides

Human THP-1 cells (ATTC) were grown in RPMI 1640 medium containing 10% fetal serum (v/v), 0.45% glucose (w/v), 10 mM Hepes, 1 mM sodium pyruvate, 1×10⁻⁵ M β-mercaptoethanol, penicillin (100 units/ml) and streptomycin (100 mg/ml). For oxidized-LDL loading experiments, cells were seeded at a density of 1×10⁶ cells/ml in medium containing 12-0-tetradecanoyl-phorbol-13-acetate (RBI) at 1×10⁻⁷ M for 24 hr. The medium was then replaced by culture medium with or without 100 μg/ml of CUSO₄ “fully” oxidized LDL (Intracel, Rockville Md.) according to the method of Hammer et al. (1995; Arterio Thromb Vasc Biol 15:704-713). Medium was replaced every two days during the time of culture. Cells were treated with Ox-LDL for 3 days. During this period, cells remained adherent and had a speckled Nile red staining pattern.

The Ox-LDL pre-treated and untreated control cells were then activated by treatment with LPS. RNA from cultured cells was prepared for expression profiling after 0, 1, and 6 hours exposure to LPS.

Total RNA was extracted using the RNA STAT-60 kit (Tel-Test, Friendswood Tex.). Poly(A) RNA was purified using the POLYATRACT mRNA isolation system (Promega). Each poly(A) RNA sample was reverse transcribed using MMLV reverse-transcriptase, 0.05 pg/μl oligo-dT primer (21mer), 1×first strand buffer, 0.03 units/ul RNase inhibitor, 500 uM dATP, 500 uM dGTP, 500 uM dTTP, 40 uM dCTP, and 40 uM either dCTP-Cy3 or dCTP-Cy5 (APB). The reverse transcription reaction was performed in a 25 ml volume containing 200 ng poly(A) RNA using the GEMBRIGHT kit (Incyte Genomics). Specific control poly(A) RNAs (YCFR06, YCFR45, YCFR67, YCFR85, YCFR43, YCFR22, YCFR23, YCFR25, YCFR44, YCFR26) were synthesized by in vitro transcription from non-coding yeast genomic DNA (W. Lei, unpublished). As quantitative controls, control mRNAs (YCFR06, YCFR45, YCFR67, and YCFR85) at 0.002 ng, 0.02 ng, 0.2 ng, and 2 ng were diluted into reverse transcription reaction at ratios of 1:100,000, 1:10,000, 1:1000, 1:100 (w/w) to sample mRNA, respectively. To sample differential expression patterns, control mRNAs (YCFR43, YCFR22, YCFR23, YCFR25, YCFR44, YCFR26) were diluted into reverse transcription reaction at ratios of 1:3, 3:1, 1:10, 10:1, 1:25, 25:1 (w/w) to sample mRNA. Reactions were incubated at 37° C. for 2 hr, treated with 2.5 ml of 0.5M sodium hydroxide, and incubated for 20 minutes at 85° C. to the stop the reaction and degrade the RNA.

Probes were purified using two successive CHROMA SPIN 30 gel filtration spin columns (Clontech Laboratories). Cy3- and Cy5-labeled reaction samples were combined as described below and ethanol precipitated using 1 ml of glycogen (1 mg/ml), 60 ml sodium acetate, and 300 ml of 100% ethanol. The probe was then dried to completion using a SpeedVAC system (Savant Instruments, Holbrook N.Y.) and resuspended in 14 μl 5×SSC/0.2% SDS.

VII. Hybridization and Detection

Hybridization reactions contained 9 μl of probe mixture consisting of 0.2 μg each of Cy3 and Cy5 labeled cDNA synthesis products from pairs of matched time point experimental and control cells in 5×SSC, 0.2% SDS hybridization buffer. The target mixture was heated to 65° C. for 5 minutes and was aliquoted onto the microarray surface and covered with an 1.8 cm² coverslip. The microarrays were transferred to a waterproof chamber having a cavity just slightly larger than a microscope slide. The chamber was kept at 100% humidity internally by the addition of 140 μl of 5×SSC in a corner of the chamber. The chamber containing the microarrays was incubated for about 6.5 hours at 60° C. The microarrays were washed for 10 min at 45° C. in low stringency wash buffer (1×SSC, 0.1% SDS), three times for 10 minutes each at 45° C. in high stringency wash buffer (0.1×SSC), and dried.

Reporter-labeled hybridization complexes were detected with a microscope equipped with an Innova 70 mixed gas 0.10 W laser (Coherent, Santa Clara Calif.) capable of generating spectral lines at 488 nm for excitation of Cy3 and at 632 nm for excitation of Cy5. The excitation laser light was focused on the microarray using a 20×microscope objective (Nikon, Melville N.Y.). The slide containing the microarray was placed on a computer-controlled X-Y stage on the microscope and raster-scanned past the objective. The 1.8 cm×1.8 cm microarray used in the present example was scanned with a resolution of 20 micrometers.

In two separate scans, the mixed gas multiline laser excited the two fluorophores sequentially. Emitted light was split, based on wavelength, into two photomultiplier tube detectors (PMT R1477; Hamamatsu Photonics Systems, Bridgewater N.J.) corresponding to the two fluorophores. Appropriate filters positioned between the microarray and the photomultiplier tubes were used to filter the signals. The emission maxima of the fluorophores used were 565 nm for Cy3 and 650 nm for Cy5. Each microarray was typically scanned twice, one scan per fluorophore using the appropriate filters at the laser source, although the apparatus was capable of recording the spectra from both fluorophores simultaneously.

The sensitivity of the scans was calibrated using the signal intensity generated by a cDNA control species. Samples of the calibrating cDNA were separately labeled with the two fluorophores and identical amounts of each were added to the hybridization mixture. A specific location on the microarray contained a complementary DNA sequence, allowing the intensity of the signal at that location to be correlated with a weight ratio of hybridizing species of 1:100,000.

The output of the photomultiplier tube was digitized using a 12-bit RTI-835H analog-to-digital (A/D) conversion board (Analog Devices, Norwood, Mass.) installed in an IBM-compatible PC computer. The digitized data were displayed as an image where the signal intensity was mapped using a linear 20-color transformation to a pseudocolor scale ranging from blue (low signal) to red (high signal). The data was also analyzed quantitatively. Where two different fluorophores were excited and measured simultaneously, the data were first corrected for optical crosstalk (due to overlapping emission spectra) between the fluorophores using each fluorophore's emission spectrum.

A grid was superimposed over the fluorescence signal image such that the signal from each spot was centered in each element of the grid. The fluorescence signal within each element was then integrated to obtain a numerical value corresponding to the average intensity of the signal. The software used for signal analysis was the GEMTOOLS gene expression analysis program (Incyte Genomics).

VIII. Data Analysis and Results

An agglomerative cluster analysis was used to identify the typical response patterns and establish the relationships between the different gene expression profiles. Each gene measurement was first normalized by dividing the expression ratios by the maximum value for each time series. To emphasize the variation from one time point to the next, slopes were added to the expression vectors by taking the expression differences between consecutive time points. The Euclidean distance was used as a similarity measure for the expression responses.

The agglomerative algorithm employed constructs a dendrogram. Starting with N clusters each containing a single gene, at each step in the iteration the two closest clusters were merged into a larger cluster. The distance between clusters was defined as the distance between their average expression patterns. After N-1 steps, all the data points were merged together. The clustering process defines a hierarchical tree. Genes were automatically assigned to a cluster by cutting the tree between the root and each gene branch with a set of 10 lines (“branch levels”) separated by fixed distances. The branch level cut-off forms a cluster. The tree was first ‘normalized’ so that each branch was at the same distance from the root. In order to preserve the distance between the closest genes, the tree was distorted at the branch furthest from the leaf. The number of branches intersecting at each branch level of the tree equals the number of clusters at that level.

Division of the tree at branch level 5 divides the 127 differentially expressed polynucleotides into 14 clusters. In tables 1, columns 4 through 10 show the level of gene expression at each time point in response to Ox-LDL exposure vs. no Ox-LDL exposure.

The OxLDL pre-treated and control cells treated with LPS and RNA were harvested at 0, 1, and 6 hrs following addition of LPS to generate six unique samples. The effect of LPS on transcription of a number of sequences encoding known and unknown proteins and other expressed polynucleotide sequences were determined in the control cells by comparing 1 hr and 6 hr RNA samples to time 0 levels (data designated: 1 h−/0− and 6 h−/0−; Table 1, columns 5 and 6, respectively). LPS effects on transcription in the OxLDL pre-treated cells were similarly determined (data designated: 1 hr+/0+ and 6 hr+/0+; Table 1, columns 7 and 8, respectively). These procedures identified the total number of LPS regulated genes in these cells. LPS responsive genes were tabulated if they exhibited a 4-fold or greater change in message levels at either 1 or 6 hrs after LPS treatment. This cut-off was chosen to select only those genes most affected by LPS treatment.

The effects of OxLDL pre-treatment was determined through an additional set of hybridization experiments. In these experiments, matched time point comparisons were made between RNA from control and OxLDL pre-treated cells after addition of LPS (data designated: 1 h+/1 h− and 6 h+/6 h−; Table 1, columns 9 and 10, respectively). This second set of hybridizations to determine the OxLDL effects on LPS responsive genes was required because only comparisons between two mRNA populations hybridized to a single chip are valid using the GEM hybridization technology. Thus, LPS responsiveness was determined through the first set of hybridizations, and OxLDL effects were determined through the second set of hybridizations. Inspection of the data for LPS response in control and OxLDL pre-treated cells shows inter-chip comparative differences; for example, the 1 h−/0− vs. 1 hr+/0+values (Table 1, columns 5 and 7, respectively) were often highly concordant with the values observed in the single chip, matched time point measurements.

IX. Complementary Nucleic Acid Molecules

Molecules complementary to the polynucleotide, or a fragment thereof, are used to detect, decrease, or inhibit gene expression. Although use of oligonucleotides comprising from about 15 to about 30 base pairs is described, the same procedure is used with larger or smaller fragments or their derivatives e.g., PNAs). Oligonucleotides are selected using OLIGO software (Molecular Insights) and SEQ ID NOs:1-127. To inhibit transcription by preventing promoter binding, a complementary oligonucleotide is designed to bind to the most unique 5′ sequence, most preferably about 10 nucleotides before the initiation codon of the open reading frame. To inhibit translation, a complementary oligonucleotide is designed to prevent ribosomal binding to the mRNA encoding the protein.

In addition to using antisense molecules constructed to interrupt transcription or translation, modifications of gene expression can be obtained by designing antisense molecules to genomic sequences (such as enhancers or introns) or even to trans-acting regulatory genes. Similarly, antisense inhibition can be achieved using Hogeboom base-pairing methodology, also known as “triple helix” base pairing. Antisense molecules involved in triple helix pairing compromise the ability of the double helix to open sufficiently for the binding of polymerases, transcription factors, or regulatory molecules.

Such antisense molecules are placed in expression vectors and used to transform preferred cells or tissues. This may include introduction of the expression vector into a cell line to test efficacy; into an organ, tumor, synovial cavity, or the vascular system for transient or short term therapy; or into a stem cell or other reproducing lineage for long term or stable gene therapy. Transient expression may last for a month or more with a non-replicating vector and for three months or more if appropriate elements for inducing vector replication are used in the transformation/expression system.

Stable transformation of appropriate dividing cells with a vector encoding the antisense molecule can produce a transgenic cell line, tissue, or organism (U.S. Pat. No. 4,736,866). Those cells that assimilate and replicate sufficient quantities of the vector to allow stable integration also produce enough antisense molecules to compromise or entirely eliminate activity of the polynucleotide.

X. Hybridization Technologies and Analyses

Hybridization technology utilizes a variety of substrates such as polymer coated glass slides and nylon membranes. Arranging elements on polymer coated slides is described in Example V; probe preparation and hybridization and analysis using polymer coated slides is described in examples VI and VII, respectively.

Polynucleotides are applied to a membrane substrate by one of the following methods. A mixture of polynucleotides is fractionated by gel electrophoresis and transferred to a nylon membrane by capillary transfer. Alternatively, the polynucleotides are individually ligated to a vector and inserted into bacterial host cells to form a library. The polynucleotides are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37° C. for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, polynucleotides are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above.

Hybridization probes derived from polynucleotides of the Sequence Listing are employed for screening cDNAs, mRNAs, or genomic DNA in membrane-based hybridizations. Probes are prepared by diluting the polynucleotides to a concentration of 40-50 ng in 45 μl TE buffer, denaturing by heating to 100° C. for five min, and briefly centrifuging. The denatured polynucleotide is then added to a REDIPRIME tube (APB), gently mixed until blue color is evenly distributed, and briefly centrifuged. Five microliters of [³²P]dCTP is added to the tube, and the contents are incubated at 37° C. for 10 min. The labeling reaction is stopped by adding 5 μl of 0.2M EDTA, and probe is purified from unincorporated nucleotides using a PROBEQUANT G-50 microcolumn (APB). The purified probe is heated to 100° C. for five min, snap cooled for two min on ice.

Membranes are pre-hybridized in hybridization solution containing 1% Sarkosyl and 1×high phosphate buffer (0.5 M NaCl, 0.1 M Na₂HPO₄, 5 mM EDTA, pH 7) at 55° C. for two hr. The probe, diluted in 15 ml fresh hybridization solution, is then added to the membrane. The membrane is hybridized with the probe at 55° C. for 16 hr. Following hybridization, the membrane is washed for 15 min at 25° C. in 1 mM Tris (pH 8.0), 1% Sarkosyl, and four times for 15 min each at 25° C. in 1 mM Tris (pH 8.0). To detect hybridization complexes, XOMAT-AR film (Eastman Kodak, Rochester N.Y.) is exposed to the membrane overnight at −70° C., developed, and examined.

XI. Expression of the Encoded Protein

Expression and purification of a protein encoded by a polynucleotide of the invention is achieved using bacterial or virus-based expression systems. For expression in bacteria, the polynucleotide is subcloned into a vector containing an antibiotic resistance gene and an inducible promoter that directs high levels of transcription. Examples of such promoters include, but are not limited to, the trp-lac (tac) hybrid promoter and the T5 or T7 bacteriophage promoter in conjunction with the lac operator regulatory element. Recombinant vectors are transformed into bacterial hosts, such as BL21(DE3). Antibiotic resistant bacteria express the protein upon induction with isopropyl beta-D-thiogalactopyranoside (IPTG). Expression in eukaryotic cells is achieved by infecting Spodoptera frugiperda (Sf9) insect cells with recombinant baculovirus, Autographica californica nuclear polyhedrosis virus. The polyhedrin gene of baculovirus is replaced with the polynucleotide by either homologous recombination or bacterial-mediated transposition involving transfer plasmid intermediates. Viral infectivity is maintained and the strong polyhedrin promoter drives high levels of polynucleotide transcription.

For ease of purification, the protein is synthesized as a fusion protein with glutathione-S-transferase (GST; APB) or a similar alternative such as FLAG. The fusion protein is purified on immobilized glutathione under conditions that maintain protein activity and antigenicity. After purification, the GST moiety is proteolytically cleaved from the protein with thrombin. A fusion protein with FLAG, an 8-amino acid peptide, is purified using commercially available monoclonal and polyclonal anti-FLAG antibodies (Eastman Kodak, Rochester N.Y.).

XII. Production of Specific Antibodies

A denatured polypeptide from a reverse phase HPLC separation is obtained in quantities up to 75 mg. This denatured protein is used to immunize mice or rabbits following standard protocols. About 100 μg is used to immunize a mouse, while up to 1 mg is used to immunize a rabbit. The denatured polypeptide is radioiodinated and incubated with murine B-cell hybridomas to screen for monoclonal antibodies. About 20 mg of polypeptide is sufficient for labeling and screening several thousand clones.

In another approach, the amino acid sequence translated from a polynucleotide of the invention is analyzed using PROTEAN software (DNASTAR) to determine regions of high immunogenicity. The optimal sequences for immunization are usually at the C-terminus, the N-terminus, and those intervening, hydrophilic regions of the polypeptide that are likely to be exposed to the external environment when the polypeptide is in its natural conformation. Typically, oligopeptides about 15 residues in length are synthesized using an 431 Peptide synthesizer (ABI) using Fmoc-chemistry and then coupled to keyhole limpet hemocyanin (KLH; Sigma Aldrich) by reaction with M-maleimidobenzoyl-N-hydroxysuccinimide ester. If necessary, a cysteine may be introduced at the N-terminus of the peptide to permit coupling to KLH. Rabbits are immunized with the oligopeptide-KLH complex in complete Freund's adjuvant. The resulting antisera are tested for antipeptide activity by binding the peptide to plastic, blocking with 1% BSA, reacting with rabbit antisera, washing, and reacting with radioiodinated goat anti-rabbit IgG.

Hybridomas are prepared and screened using standard techniques. Hybridomas of interest are detected by screening with radioiodinated polypeptide to identify those fusions producing a monoclonal antibody specific for the polypeptide. In a typical protocol, wells of 96 well plates (BD Biosciences, San Jose Calif.) are coated with affinity-purified, specific rabbit-anti-mouse (or suitable anti-species Ig) antibodies at 10 mg/ml. The coated wells are blocked with 1% BSA and washed and exposed to supernatants from hybridomas. After incubation, the wells are exposed to radiolabeled polypeptide at 1 mg/ml. Clones producing antibodies bind a quantity of labeled polypeptide that is detectable above background.

Such clones are expanded and subjected to 2 cycles of cloning at 1 cell/3 wells. Cloned hybridomas are injected into pristane-treated mice to produce ascites, and monoclonal antibody is purified from the ascitic fluid by affinity chromatography on protein A (APB). Monoclonal antibodies with affinities of at least 10⁸ M⁻¹, preferably 10⁹ to 10¹⁰ M⁻¹ or stronger, are made by procedures well known in the art.

XIII. Purification of Naturally Occurring Protein Using Specific Antibodies

Naturally occurring or recombinant protein is purified by immunoaffinity chromatography using antibodies specific for the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIV. Screening Molecules for Specific Binding

The polynucleotide or fragments thereof are labeled with ³²P-dCTP, Cy3-dCTP, Cy5-dCTP (APB), or the protein or portions thereof are labeled with BIODIPY or FITC (Molecular Probes). A library or a plurality of candidate molecules or compounds previously arranged on a substrate are incubated in the presence of labeled polynucleotide or protein. After incubation under conditions for a polynucleotide or protein, the substrate is washed. Any position on the substrate retaining label, that indicates specific binding or complex formation, identifies a ligand. Data obtained using different concentrations of the polynucleotide or polypeptide are used to calculate affinity between the labeled polynucleotide or protein and the bound ligand.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

TABLE 1

| SEQ ID NO | Incyte ID | Sequence Annotation | 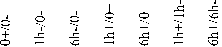 |
|---|---|---|---|
| 1 | 460023.9 | Human calmodulin (CALM2) gene, exons 3-6, and complete cds | 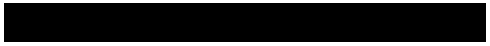 |
| 2 | 1004532.45 | Human mRNA for KIAA0930 protein, partial cds. | 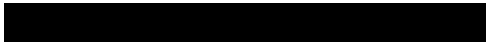 |
| 3 | 1383324.156 | Human mRNA for beta-adrenergic receptor kinase. | 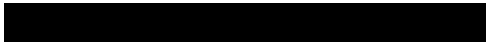 |
| 4 | 1400053CB1 | leukotriene A4 hydrolase | 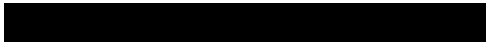 |
| 5 | 2874030CB1 | Human differentiation antigen (CD33) mRNA, complete cds. | 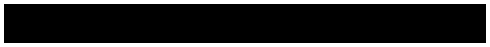 |
| 6 | 2049950CB1 | glutamate-cysteine ligase (gamma-glutamylcysteine synthetase), catalytic (72.8kD) | 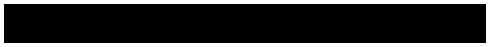 |
| 7 | 3257507CB1 | ref-1 = redox factor [Human, mRNA, 1441 nt]. | 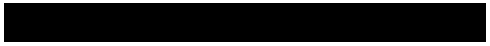 |
| | | Human TTG-2 mRNA for a cysteine rich protein with LIM motif. | 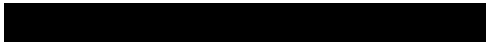 |
| 8 | 2676869CB1 | Human CGI-142 protein mRNA, complete cds. | 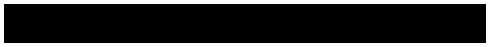 |
| 9 | 672615CB1 | Human microsomal glutathione S-transferase 2 (MGST2) mRNA, complete cds. | 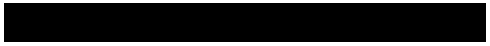 |
| | | Human H-cadherin mRNA, complete cds. | 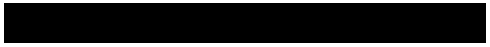 |
| | | Human adenylyl cyclase-associated protein (CAP) mRNA, complete cds. | 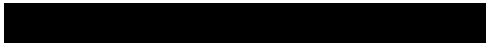 |
| 10 | 1385861.1 | Human G-protein-coupled receptor V28 mRNA, complete cds. | 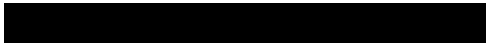 |
| 11 | 1383798.8 | embigin protein | 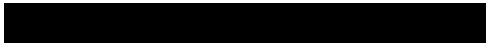 |
| | | macrophage inhibitory cytokine-1 | 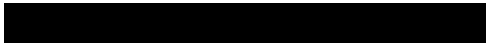 |
| | | Human cellular proto-oncogene (c-mer) mRNA, complete cds. | 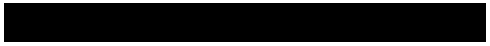 |
| 12 | 2616727CB1 | LIM and SH3 protein 1 (MLN50) | 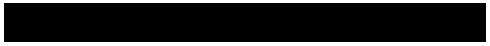 |
| | | Human (HeLa) small nuclear U5 A RNA. | 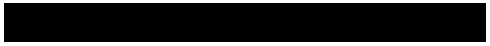 |
| 13 | 251906.2 | Human CCAAT/enhancer binding protein alpha gene, complete cds. | 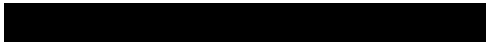 |
| 14 | 166400.36 | Human K+ channel beta 2 subunit mRNA, complete cds. | 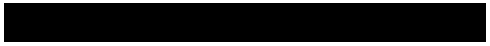 |
| 15 | 1354395CB1 | RhoGAP | 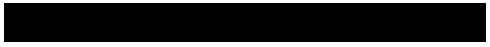 |
| | | Human p55CDC mRNA, complete cds. | 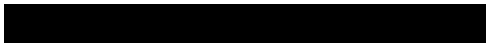 |
| | | Human insulin induced protein 1 (INSIG1) gene, complete cds. | 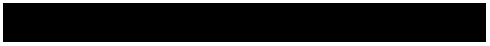 |
| | | insulin induced gene 1 | 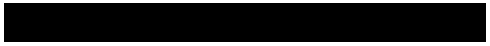 |
| | | Human chemokine (hmrp-2a) mRNA, complete cds. | 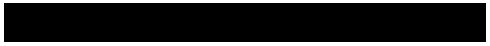 |
| | | gene predicted from cDNA with a complete coding sequence | 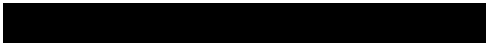 |
| | | Human mRNA; cDNA DKFZp586N041 (from clone DKFZp586N041). | 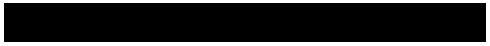 |
| 16 | 253541.1 | Human protein tyrosine phosphatase mRNA, complete cds. | 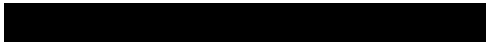 |
| | | Human plasma membrane calcium ATPase isoform 1 (ATP2B1) gene | 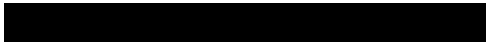 |
| 17 | 1966280CB1 | Human mRNA for pentaxin (PTX3). | 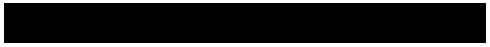 |
| | | Human plasma membrane calcium ATPase isoform 1 (ATP2B1) gene | 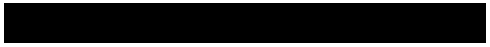 |
| | | Nijmegen breakage syndrome 1 (nibrin) | 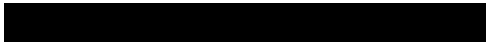 |
| | | SWI/SNF related, matrix associated, actin dependent regulator of chromatin, subfamily a, member 5 | 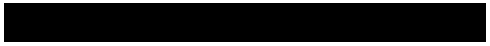 |
| 18 | 1399416.4 | ESTs | 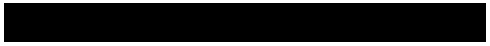 |
| 19 | 1250434CB1 | hypoxia-inducible factor 1, alpha subunit (basic helix-loop-helix transcription factor) | 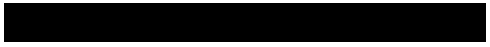 |
| 20 | 1377369.2 | spermidine/spermine N1-acetyltransferase | 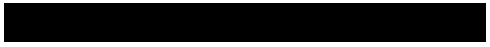 |
| 21 | 342012CB1 | B-cell translocation gene 1, anti-proliferative | 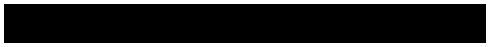 |
| 22 | 1438184CB1 | nuclear factor of kappa light polypeptide gene enhancer in B-cells 1 (p105) | 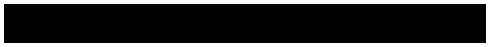 |
| 23 | 1450758CB1 | Human MRS1 mRNA, complete cds. | 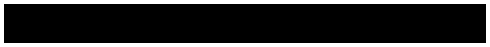 |
| 24 | 1841778CB1 | Human mRNA for interleukin-1 receptor antagonist. | 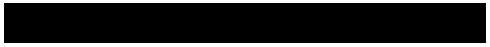 |
| 25 | 2814863CB1 | pre-B-cell colony-enhancing factor | 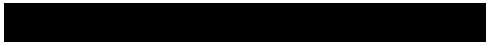 |
| | | fatty-acid-Coenzyme A ligase, long-chain 1 | 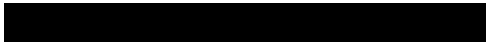 |
| 26 | 1218366CB1 | pleckstrin | 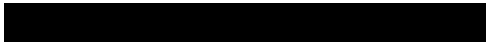 |
| | | Human G0S2 protein gene, complete cds | 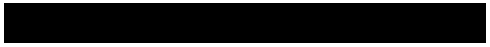 |
| 27 | 2483854CB1 | interleukin 1, alpha | 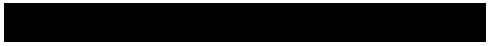 |
| | | Human mRNA for pleckstrin (P47). | 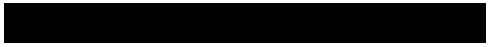 |
| 28 | 1842870CB1 | myeloid cell leukemia sequence 1 (BCL2-related) | 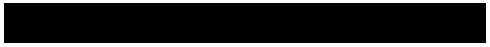 |
| 29 | 235631.1 | dual specificity phosphatase 2 | 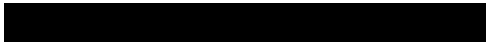 |
| 30 | 156986CB1 | melibiose carrier protein | 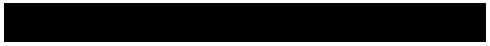 |
| 31 | 1136056.2 | Human G0S3 mRNA, complete cds. | 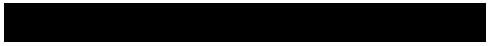 |
| 32 | 3732868CB1 | Human G0S2 gene, 5′ flank and cds. | 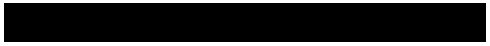 |
| | | Human mRNA for interleukin-1 precursor (pre IL-1). | 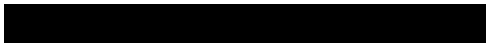 |
| 33 | 1528186CB1 | Human TRAF-interacting protein I-TRAF mRNA, complete cds. | 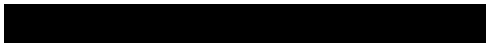 |
| 34 | 1453334CB1 | plasminogen activator, urokinase | 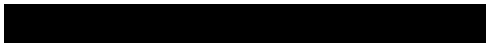 |
| 35 | 779175CB1 | FOS-like antigen 2 | 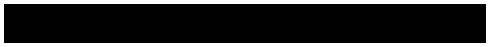 |
| 36 | 246574.3 | Human gro (growth regulated) gene. | 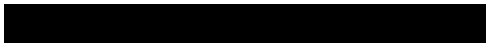 |
| | | Human protein tyrosine phosphatase (PAC-1) mRNA, complete cds. | 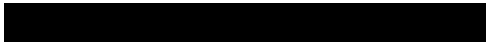 |
| 37 | 227742.4 | ESTs | 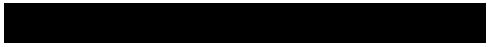 |
| | | small inducible cytokine subfamily A (Cys-Cys), memb 18, pulmonary and activation-regulated | 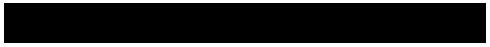 |
| | | small inducible cytokine subfamily A (Cys-Cys), memb 18, pulmonary and activation-regulated | 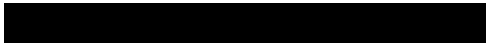 |
| 38 | 337792.2 | phorbol-12-myristate-13-acetate-induced protein 1 | 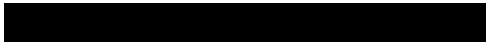 |
| | | KIAA0212 gene product | 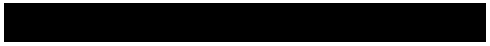 |
| 39 | 57385.565 | Human EBV induced G-protein coupled receptor (EBI2) mRNA, complete cds. | 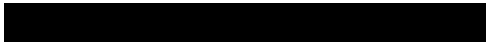 |
| 40 | 331150.1 | interleukin 10 receptor, alpha | 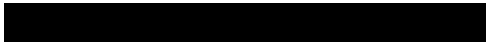 |
| 41 | 989010.6 | Human monocyte chemotactic protein gene, complete cds. | 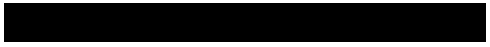 |
| | | CD44 | 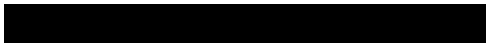 |
| 42 | 3072333CB1 | insulin-like growth factor binding protein 3 | 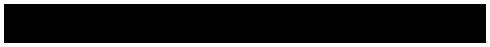 |
| 43 | 247025.3 | chemokine (C-C motif) receptor 7 | 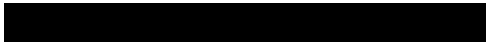 |
| 44 | 229299.3 | small inducible cytokine subfamily B (Cys-X-Cys), member 11 | 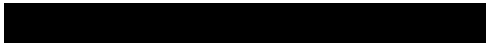 |
| 45 | 1452055CB1 | tumor necrosis factor receptor superfamily, member 5 | 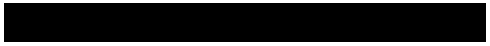 |
| 46 | 410442.8 | Human ETS oncogene (PEP1) mRNA, complete cds. | 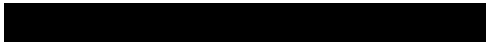 |
| 47 | 1518310CB1 | Human connexin 26 (GJB2) mRNA, complete cds. | 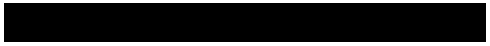 |
| 48 | 405646.17 | tumor necrosis factor receptor superfamily, member 5 | 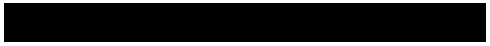 |
| | | Human interferon-induced cellular resistance mediator protein (MxB) mRNA, complete cds. | 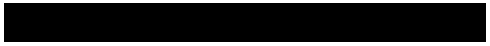 |
| 49 | 1136376.164 | Human mRNA for 80K-L protein, complete cds. | 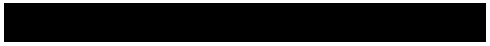 |
| 50 | 233104.1 | ESTs | 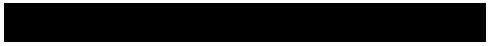 |
| 51 | 2303994CB1 | Mpc1 locus, uterus and prostate cancer-associated | 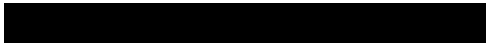 |
| | | Incyte EST | 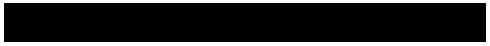 |
| | | Human lyn mRNA encoding a tyrosine kinase. | 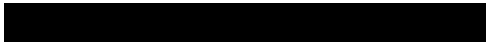 |
| 52 | 376673.3 | IFN-induced polynucleotide | 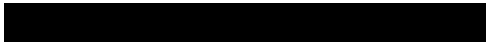 |
| 53 | 346511.1 | 2′-5′oligoadenylate synthetase 2 | 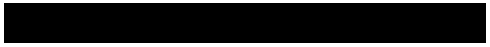 |
| | | Human interferon-induced cellular resistance mediator protein (MxA) mRNA, complete cds. | 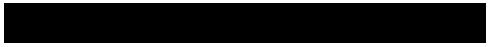 |
| 54 | 1554082CB1 | GRAIL | 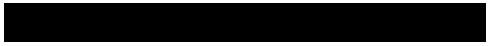 |
| 55 | 5185743CB1 | Human tonicity-responsive enhancer-binding protein mRNA, complete cds. | 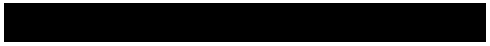 |
| 56 | 899263.12 | KIAA0032 | 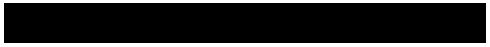 |
| | | Human (2′-5′) oligo A synthetase E gene, exon 7 and flanks. | 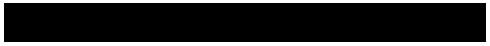 |
| 57 | 218602.1 | ubiquitin specific protease UBP43 | 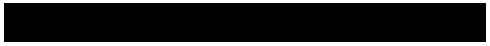 |
| 58 | 4436929CB1 | Human HPK/GCK-like kinase HGK mRNA, complete cds. | 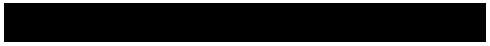 |
| 59 | 222981.14 | Human proto-oncogene (BCL3) gene, exons 3-9 and complete cds. | 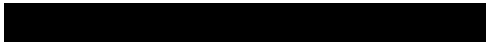 |
| | | ring finger protein | 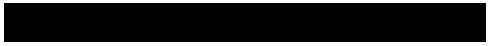 |
| 60 | 1453496.9 | Human transcription factor ISGF-3 mRNA, complete cds. | 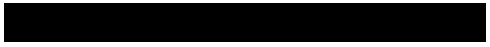 |
| 61 | 020293CB1 | Human B lymphocyte chemoattractant BLC mRNA, complete cds. | 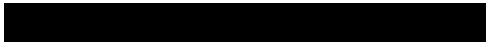 |
| | | small inducible cytokine subfamily B (Cys-X-Cys), memb 5 (epithelial-derived neutrophil-activating peptide 78) | 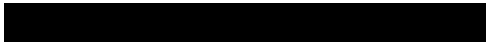 |
| 62 | 135626CB1 | Human phorbolin-1-related protein mRNA, complete cds. | 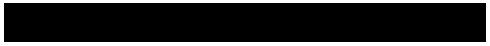 |
| 63 | 244262.2 | small inducible cytokine subfamily B (Cys-X-Cys), memb 6 (granulocyte chemotactic protein 2) | 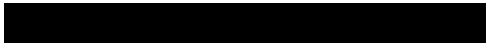 |
| | | Human myeloid cell differentiation protein (MCL1) mRNA. | 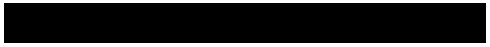 |
| | | Human endoperoxide synthase type II mRNA, complete cds. | 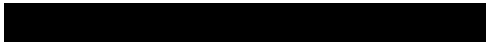 |
| 64 | 1330247.162 | Human rhoB gene mRNA. | 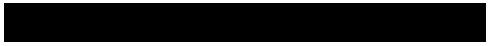 |
| 65 | 1377892.2 | regulator of G-protein signalling 2, 24kD | 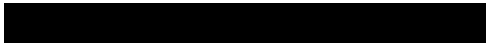 |
| 66 | 1446506.1 | MAIL nuclear I kappa B protein | 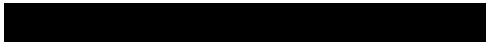 |
| 67 | 53959.1 | Human zinc finger transcriptional regulator mRNA, complete cds. | 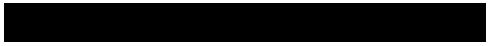 |
| 68 | 223092.1 | tumor necrosis factor (TNF superfamily, member 2) | 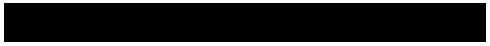 |
| | | GTP-binding protein overexpressed in skeletal muscle | 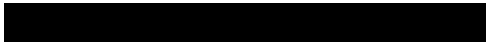 |
| 69 | 335430.4 | Human mRNA for leucine-rich primary response protein 1. | 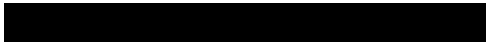 |
| | | Human Gem GTPase (gem) mRNA, complete cds. | 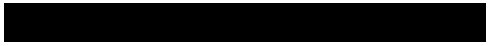 |
| 70 | 1398075.7 | Jun activation domain binding protein | 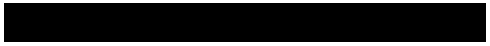 |
| | | Human activating transcription factor 3 (ATF3) mRNA, complete cds. | 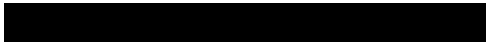 |
| 71 | 1453496.8 | Homo sapiens transcription factor ISGF-3 mRNA, complete cds | 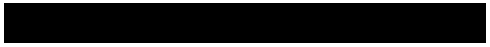 |
| | | Human transcription factor ISGF-3 mRNA, complete cds. | 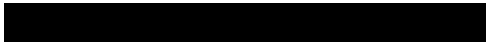 |
| | | aquaporin 9 | 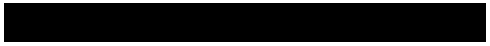 |
| | | Human neutrophil oxidase factor (p67-phox) mRNA, complete cds. | 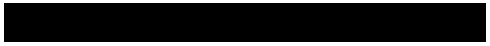 |
| 72 | 1327351.406 | Human cell adhesion molecule (CD44) mRNA, complete cds. | 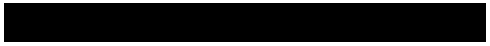 |
| 73 | 200578.1 | ESTs | 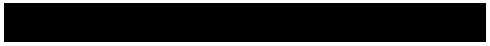 |
| 74 | 2835028CB1 | interleukin 7 receptor | 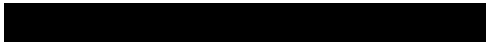 |
| | | Fig-1 protein | 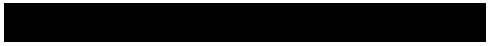 |
| 75 | 2875023CB1 | kynureninase (L-kynurenine hydrolase) | 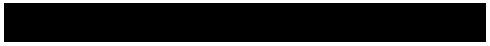 |
| 76 | 3173735CB1 | guanylate binding protein 1, interferon-inducible, 67kD | 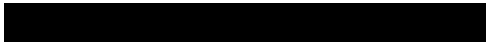 |
| 77 | 474301.1 | Human mRNA for KIAA0942 protein, partial cds. | 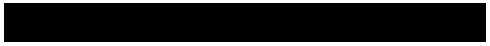 |
| 78 | 406992.1 | cystine/glutamate transporter | 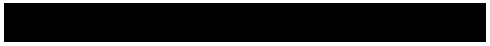 |
| | | CD44 antigen (homing function and Indian blood group system) | 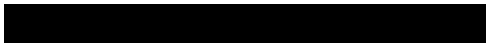 |
| 79 | 234681.21 | cardiac/skeletal muscle M5-14 | 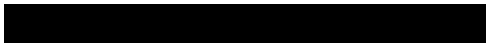 |
| 80 | 409194.1 | Human Ig rearranged B7 protein mRNA VC1-region, complete cds. | 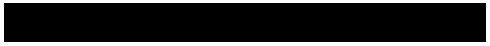 |
| 81 | 258971CB1 | interferon-induced protein 56 | 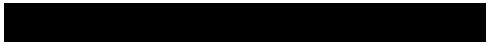 |
| 82 | 231486.27 | Human gene for hepatitis C-associated microtubular aggregate protein p44, exon 8. | 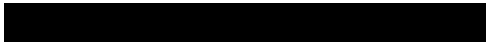 |
| 83 | 1930967CB1 | Human semaphorin-III (Hsema-I) mRNA, complete cds. | 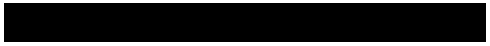 |
| 84 | 279117.32 | Human phospholipid scramblase mRNA, complete cds. | 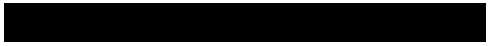 |
| 85 | 898547.1 | proline-rich post-synaptic synaps-associated protein PDZ | 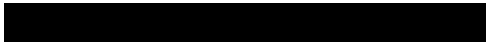 |
| 86 | 202212.16 | Human TNF-related apoptosis inducing ligand TRAIL mRNA, complete cds. | 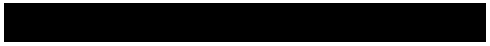 |
| 87 | 2874529CB1 | protein kinase, interferon-inducible double stranded RNA dependent | 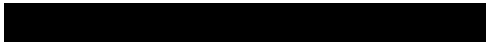 |
| | | Human 69 kDa 2′5′ oligoadenylate synthetase (P69 2-5A synthetase) mRNA, complete cds. | 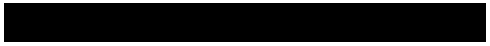 |
| | | Human G-protein-coupled receptor (EBI 1) mRNA, complete cds. | 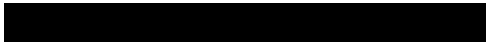 |
| 88 | 2705515CB1 | tryptophanyl-tRNA synthetase | 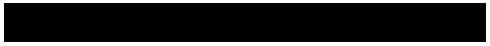 |
| | | interferon stimulated gene (20kD) | 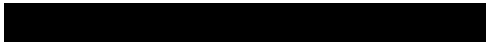 |
| 89 | 1328372.14 | Human interferon-inducible protein 9-27 mRNA, complete cds. | 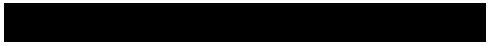 |
| 90 | 253534.2 | myxovirus (influenza) resistance 1, homolog of murine (interferon-inducible protein p78) | 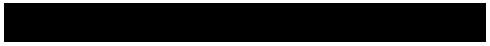 |
| 91 | 082155CB1 | stimulated trans-acting factor (50 kDa) | 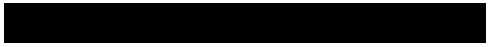 |
| | | small inducible cytokine subfamily A (Cys-Cys), memb 8 (monocyte chemotactic protein 2) | 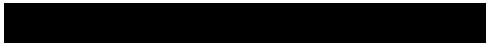 |
| 92 | 1924205CB1 | myxovirus (influenza) resistance 2, homolog of murine | 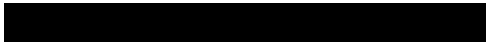 |
| 93 | 965517CB1 | Human mRNA for monocyte chemotactic protein-2. | 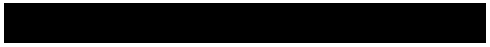 |
| 94 | 1795309CB1 | Human HEM45 mRNA, complete cds. | 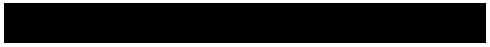 |
| | | Human insulin-like growth factor-binding protein-3 gene, complete cds, clone HL1006d. | 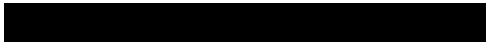 |
| 95 | 816792CB1 | Human vascular endothelial cell growth factor 165 receptor 2 (VEGF165R2) mRNA, complete cds. | 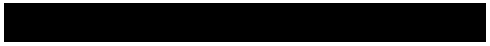 |
| 96 | 3838440CB1 | Human ubiquitin hydrolyzing enzyme I (UBH1) mRNA, partial cds. | 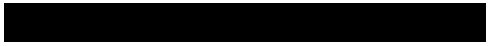 |
| 97 | 3282941CB1 | Human activated B-cell factor-1 (ABF-1) mRNA, complete cds. | 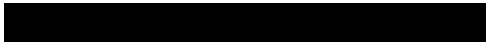 |
| 98 | 1399366.28 | Human mRNA for thrombospondin. | 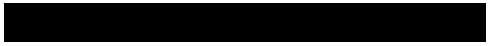 |
| 99 | 269059.41 | connective tissue growth factor | 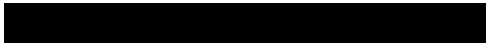 |
| | | Human metallothionein-II pseudogene (mt-lips). | 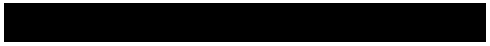 |
| | | immediate early response 3 | 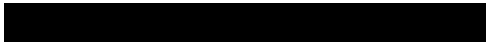 |
| | | inositol 1,4,5-triphosphate receptor, type 1 | 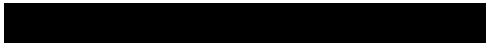 |
| 100 | 5511889CB1 | Down syndrome candidate region 1 | 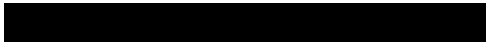 |
| 101 | 1671522CB1 | Human mRNA for NOT. | 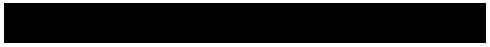 |
| 102 | 1447015.4 | retinal methyl CpG-binding PCM-1; pilot | 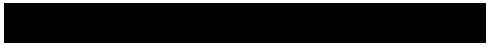 |
| 103 | 1306684.34 | nuclear receptor subfamily 4, group A, member 1 | 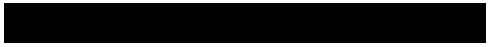 |
| 104 | 440806.3 | Human pilot mRNA. | 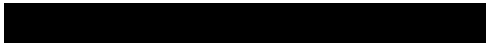 |
| 105 | 3493433 | nuclear receptor subfamily 4, group A, member 3 | 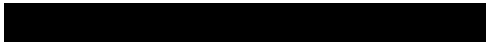 |
| | | adipose differentiation-related protein; adipophilin | 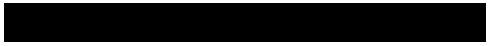 |
| | | Human antigen CD36 (clone 13) mRNA, complete cds. | 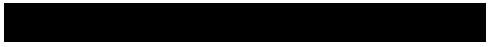 |
| | | Homo sapiens mRNA for zinc finger protein, complete cds | 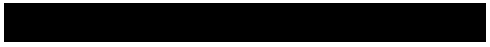 |
| | | Human mRNA for P2 protein of peripheral myelin. | 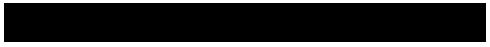 |
| | | apolipoprotein C-I | 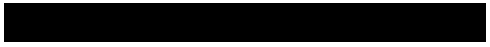 |
| | | Human mRNA for white gene protein. | 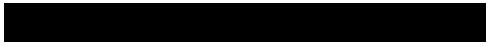 |
| | | Human lipoprotein lipase mRNA, complete cds. | 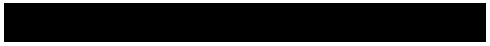 |
| | | Human pyruvate dehydrogenase kinase isoform 4 mRNA, complete cds. | 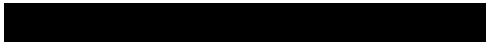 |
| | | Human TNFR-related death receptor-6 (DR6) mRNA, complete cds. | 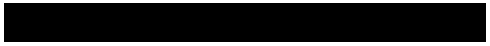 |
| | | Human fatty acid binding protein homologue (PA-FABP) mRNA, complete cds. | 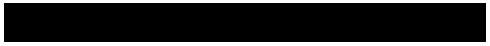 |
| | | Human ECRP gene for eosinophil cationic related protein | 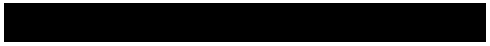 |
| 106 | 277161.34 | CD-20-like precursor, dendritic cell HAIRB | 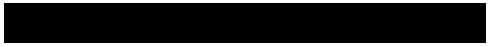 |
| | | allograft inflammatory factor 1 | 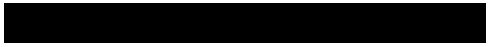 |
| | | ferritin, heavy polypeptide 1 | 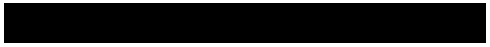 |
| | | Human ferritin heavy-chain gene, exons 2, 3 and 4. | 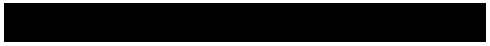 |
| 107 | 2588008CB1 | regulator of G-protein signalling 1 | 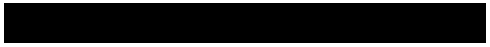 |
| 108 | 1794550CB1 | ras homolog gene family, member E | 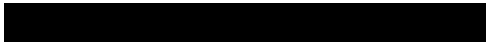 |
| | | RhoE=26 kda GTPase homolog [Human, HeLa cell line, mRNA, 833 nt]. | 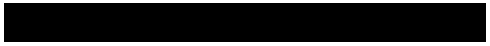 |
| | | Human glia-derived nexin (GDN) mRNA, 5′ end. | 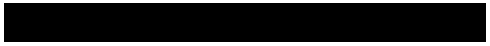 |
| | | Human glia-derived nexin (GDN) mRNA, 5′ end. | 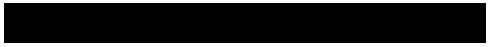 |
| 109 | 75272.34 | upregulated by 1,25-dihydroxyvitamin D-3 | 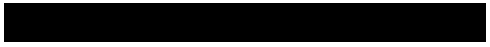 |
| | | interferon (gamma)-induced cell line; protein 10 from | 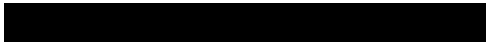 |
| 110 | 236030.3 | Human mRNA for gamma-interferon inducible early response gene (with homology to platelet proteins). | 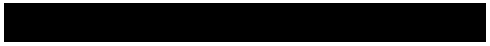 |
| 111 | 1749102CB1 | indoleamine-pyrrole 2,3 dioxygenase | 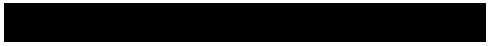 |
| | | Human cell-line THP-1 small cytokine B subfamily member 11 SCYB11 precursor, mRNA, complete cds. | 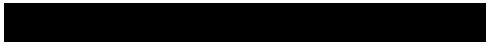 |
| | | Human interferon-gamma-inducible indoleamine 2,3-dioxygenase (IDO) mRNA, complete cds. | 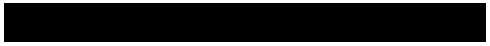 |
| | | Human interleukin-7 receptor (IL-7) mRNA, complete cds. | 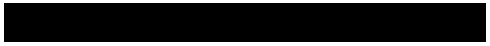 |
| 112 | 003360CB1 | Human mRNA for pLD78 peptide, complete cds. | 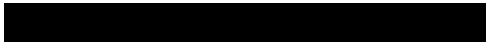 |
| 113 | 1398150.29 | small inducible cytokine A4 (homologous to mouse Mip-1b) | 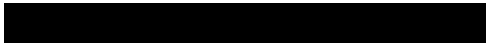 |
| 114 | 088564CB1 | Human chemokine exodus-1 mRNA, complete cds. | 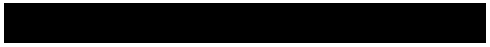 |
| | | Human gene for prointerleukin 1 beta. | 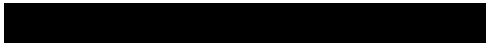 |
| | | small inducible cytokine subfamily A (Cys-Cys), member 20 | 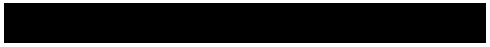 |
| | | Human homologue-2 of gene encoding alpha subunit of murine cytokine (MIP1/SCI), complete cds. | 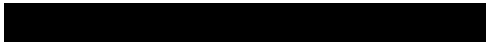 |
| 115 | 149431.7 | interleukin 1, beta | 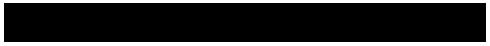 |
| 116 | 1750533CB1 | Human tumor necrosis factor alpha inducible protein A20 mRNA, complete cds. | 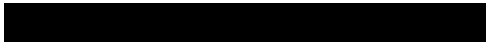 |
| | | tumor necrosis factor, alpha-induced protein 3 | 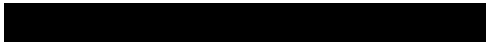 |
| 117 | 1324237.8 | Human cytokine (GRO-beta) mRNA, complete cds. | 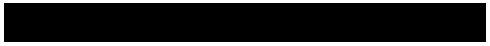 |
| 118 | 1283029.16 | Human CL 100 mRNA for protein tyrosine phosphatase. | 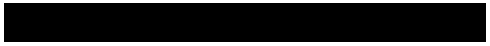 |
| 119 | 128475.1 | Incyte EST; mouse spleen MAIL, nuclear 1-kappaB potentiated by LPS | 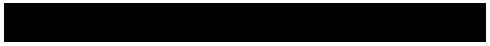 |
| 120 | 561301CB1 | Human mRNA for tumor necrosis factor. | 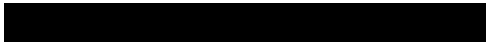 |
| 121 | 828082CB1 | activating transcription factor 3 | 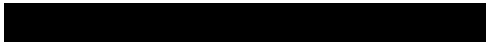 |
| | | superoxide dismutase 2, mitochondrial | 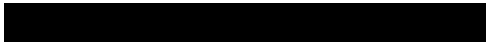 |
| 122 | 1097190.34 | Human mRNA for Mn superoxide dismutase (EC 1.15.1.1.). | 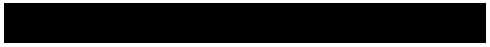 |
| | | BCL2-related protein A1 | 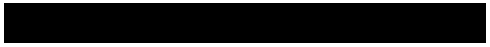 |
| 123 | 637393CB1 | Human mRNA for GRS protein. | 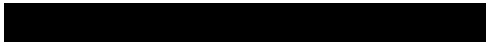 |
| 124 | 191918CB1 | tumor necrosis factor, alpha-induced protein 6 | 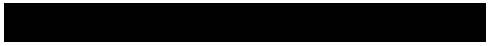 |
| | | Human TNF-inducible (TSG-6) mRNA fragment, adhesion receptor CD44 putative cds. | 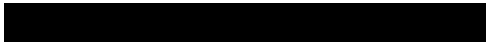 |
| 125 | 154697CB1 | interferon-induced protein 54 | 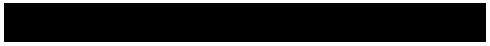 |
| 126 | 271804.3 | prostaglandin-endoperoxide synthase 2 (prostaglandin G/H synthase and cyclooxygenase) | 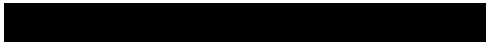 |
| | | Human beta-thromboglobulin-like protein mRNA, complete cds. | 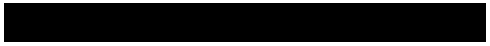 |
| | | Human monocyte interleukin 1 (IL-1) mRNA, complete cds. | 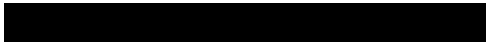 |
| 128 | 1453257.6 | interleukin 8 | 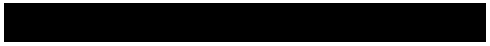 |
| | | peripheral myelin protein 2 | 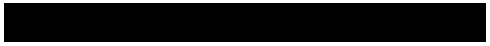 |
| | | fatty acid binding protein 4, adipocyte | 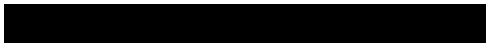 |

TABLE 2

| SEQ ID | SEQ ID |
|---|---|
| NO | NO |
| 4 | 128 |
| 5 | 129 |
| 6 | 130 |
| 7 | 131 |
| 8 | 132 |
| 9 | 133 |
| 12 | 134 |
| 15 | 135 |
| 17 | 136 |
| 19 | 137 |
| 21 | 138 |
| 22 | 139 |
| 23 | 140 |
| 24 | 141 |
| 25 | 142 |
| 26 | 143 |
| 27 | 144 |
| 28 | 145 |
| 30 | 146 |
| 32 | 147 |
| 33 | 148 |
| 34 | 149 |
| 35 | 150 |
| 42 | 151 |
| 45 | 152 |
| 47 | 153 |
| 51 | 154 |
| 54 | 155 |
| 55 | 156 |
| 58 | 157 |
| 61 | 158 |
| 62 | 159 |
| 74 | 160 |
| 75 | 161 |
| 76 | 162 |
| 81 | 163 |
| 83 | 164 |
| 87 | 165 |
| 88 | 166 |
| 91 | 167 |
| 92 | 168 |
| 93 | 169 |
| 94 | 170 |
| 95 | 171 |
| 96 | 172 |
| 97 | 173 |
| 100 | 174 |
| 101 | 175 |
| 107 | 176 |
| 108 | 177 |
| 111 | 178 |
| 112 | 179 |
| 114 | 180 |
| 116 | 181 |
| 120 | 182 |
| 121 | 183 |
| 123 | 184 |
| 124 | 185 |
| 125 | 186 |

What is claimed is:

1. A combination comprising a plurality of polynucleotides wherein the polynucleotides are SEQ ID NOs:1-127 and the complements of SEQ ID NOs:1-127.

2. The combination of claim 1, wherein each of the polynucleotides is differentially expressed in LPS-treated foam cells and is selected from:
a) SEQ ID NOs:16-105 and 109-127;
b) SEQ ID NOs:1-15 and 106-108; and
c) a complement of (a) or (b).

3. The combination of claim 1, wherein each of the polynucleotides is differentially expressed in LPS-treated foam cells and is selected from:
a) SEQ ID NOs:16-25, 50-63, and 71-88, and 109-111;
b) SEQ ID NO:26-38;
c) SEQ ID NOs:65-70, 100-105, 112-121;
d) SEQ ID NOs:122-127;
e) SEQ ID NOs:1-11 and 106-108; and
f) the complements of (a), (b), (c), (d), or (e).

4. The combination of claim 1, wherein the polynucleotides are immobilized on a substrate.

5. A high throughput method for detecting differential expression of one or more polynucleotides in a sample, the method comprising:
a) hybridizing the combination of claim 2 with the sample, thereby forming one or more hybridization complexes;
b) detecting the hybridization complexes; and
c) comparing the hybridization complexes with those of a standard, wherein each difference in the size and intensity of a hybridization complex indicates differential expression of a polynucleotide in the sample.

6. The method of claim 5, wherein the sample is from a subject with atherosclerosis and comparison with a standard defines early, mid, or late stages of the disorder.

7. A high throughput method of screening a library of molecules or compounds to identify a ligand which binds a polynucleotide, the method comprising:
a) combining the combination of claim 1 with the library under conditions to allow specific binding; and
b) detecting specific binding between the polynucleotide and a molecule or compound, thereby identifying a ligand that specifically binds to the polynucleotide.

8. The method of claim 7 wherein the library is selected from DNA molecules, peptides, proteins and RNA molecules.

9. A method of obtaining an extended or full length gene from a library of nucleic acid sequences, the method comprising:
a) arranging individual sequences on a substrate;
b) hybridizing a polynucleotide of claim 1 with the sequences under conditions which allow specific binding;
c) detecting hybridization between the polynucleotide and one or more sequences; and
d) isolating the sequences from the library, thereby obtaining extended or full length gene.

10. A purified polynucleotide having a nucleic acid sequence selected from SEQ ID NOs:51, 52, 54, 79, 85, 102, 106, and 119 or the complements of SEQ ID NOs:51, 52, 54, 79, 85, 102, 106, and 119.

11. An expression vector containing the polynucleotide of claim 10.

12. A host cell containing the expression vector of claim 11.

13. A purified polypeptide comprising an amino acid sequence of SEQ ID NOs:154 or 155.

14. A method for producing a protein, the method comprising the steps of:
a) culturing the host cell of claim 12 under conditions for the expression of protein; and
b) recovering the protein from the host cell culture.

15. A protein produced by the method of claim 14.

16. A high-throughput method for screening a library of molecules or compounds to identify at least one ligand which specifically binds a protein, the method comprising:
a) combining the protein or a portion thereof of claim 15 with the library under conditions to allow specific binding; and
b) detecting specific binding between the protein and a molecule or compound, thereby identifying a ligand which specifically binds the protein.

17. A method of purifying a ligand from a sample, the method comprising:
a) combining the protein of claim 15 with a sample under conditions to allow specific binding;
b) recovering the bound protein; and
c) separating the protein from the ligand, thereby obtaining purified ligand.

18. A method of making a antibody, the method comprising:
a) immunizing an animal with the protein of claim 15 under conditions to elicit an antibody response,
b) isolating animal antibodies, and
c) screening the isolated antibodies with the protein to identify an antibody that specifically binds the protein.

19. A composition comprising the protein of claim 15.

20. A purified antibody that specifically binds to the protein of claim 15.
